SEQUENCE LISTING (3 sequences)

SEQ ID NO: 1 - protein, 28 aa, Artificial Sequence
  synthetic peptide; N-terminal end of PPARgamma variant 2
MGETLGDSPIDPESDSFTDTLSANISQE

SEQ ID NO: 2 - protein, 25 aa, Artificial Sequence
  synthetic peptide; NCoR1 residues 2251-2275
GHSFADPASNLGLEDIIRKALMGSF

SEQ ID NO: 3 - protein, 22 aa, Artificial Sequence
  synthetic peptide; SMRT residues 1316-1337
TNMGLEAIIRKALMGKYDQWEE

(19) United States
(12) Patent Application Publication
(10) Pub. No.: US 2013/0158063 A1
(43) Pub. Date: Jun. 20, 2013

(54) Compounds, Compositions and Methods Related to PPAR Antagonists

(71) Applicants: Milton Lang Brown, Brooksville, MD (US); Yali Kong, Centreville, VA (US); Yong Liu, Rockville, MD (US); Robert Glazer, Potomac, MD (US); York Tomita, Bethesda, MD (US)

(72) Inventors: Milton Lang Brown, Brooksville, MD (US); Yali Kong, Centreville, VA (US); Yong Liu, Rockville, MD (US); Robert Glazer, Potomac, MD (US); York Tomita, Bethesda, MD (US)

(73) Assignee: GEORGETOWN UNIVERSITY, Washington, DC (US)

(21) Appl. No.: 13/818,966

(22) PCT Filed: Aug. 24, 2011
(86) PCT No.: PCT/US11/48981

§ 371 Date: Feb. 25, 2013

Related U.S. Application Data
(60) Provisional application No. 61/376,600, filed on Aug. 24, 2010.

(51) Int. Cl. C07C 311/51; C07D 471/04; C07D 263/48

(52) U.S. Cl. CPC C07C 311/51; C07D 263/48; C07D 471/04
USPC 514/285; 564/81; 514/604; 548/233; 514/377; 546/70

(57) ABSTRACT

Disclosed are compounds, compositions and methods related PPAR antagonists. Certain compounds are effective at inhibiting PPARs. The compositions can be used to inhibit PPARs, treat cancer and treat metabolic disorders.

### II. CROSS-REFERENCE TO RELATED APPLICATIONS

This application claims benefit of U.S. Provisional Application No. 61/376,600, filed Aug. 24, 2010. Application No. 61/376,600, filed Aug. 24, 2010, is hereby incorporated herein by reference in its entirety.

### I. STATEMENT REGARDING FEDERALLY SPONSORED RESEARCH

This invention was made with government support under Grant No. FBS-43312-64 awarded to Thermo-Fisher Bioservices, Inc. and awarded by the National Cancer Institute (NCI) of the National Institutes of Health (NIH). The government has certain rights in the invention.

### III. REFERENCE TO SEQUENCE LISTING

The Sequence Listing submitted Aug. 24, 2011 as a text file named “GU_{—}18_{—}9001_AMD_AFD_Sequence_Listing_Text_File.txt,” created on Aug. 23, 2011, and having a size of 1,366 bytes is hereby incorporated by reference pursuant to 37 C.F.R. §1.52(e)(5).

### IV. BACKGROUND

Nuclear receptors represent an important class of receptor targets for drug discovery. The peroxisome proliferator-activated receptors (PPARs) are ligand activated transcription factors that belong to the nuclear receptor superfamily and play very important roles in multiple physiological pathways. Three PPAR receptor subtypes with distinct tissue distributions, designated as PPARα, PPARγ and PPARβ/δ, have been identified. The PPARs coordinate pathways involved in glucose and lipid homeostasis (Willson M. T. et al. J Med Chem 43:527-550, 2000; Berger J. et al. Annu Rev Med 53:409-435, 2002). In addition, PPARγ and PPARβ/δ are involved in developmental and differentiation pathways and therefore play important roles in embryogenesis, inflammation and cancer (Zaveri, T. N. et al. Canc Biol Ther 8:1252-1261, 2009; Elikkottil, J. et al. Canc Biol Ther 8:1262-1264, 2009).

### V. SUMMARY

Disclosed herein are compounds, compositions and methods. The compounds, compositions and methods are antagonists of peroxisome proliferator-activated receptors (PPARs).

Disclosed herein are compounds having the structure:

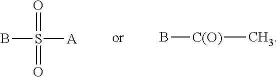

In some forms, the compounds, compositions and methods relate to inhibiting PPARs. In some forms, the compounds, compositions and methods relate to treatment of cancer or metabolic disorders.

The objects, advantages and features of the compounds, compositions and methods disclosed herein will become more apparent when reference is made to the following description taken in conjunction with the accompanying drawings.

### VI. BRIEF DESCRIPTION OF FIGURES

FIG. 1 shows the structure of PPAR antagonists and biological data of YL-1-04-02. A) BTB07995 and its derivatives. B) Fluorescent spectra of YL-1-04-02.

FIG. 2 shows a PPAR reporter assay for compounds structurally related to YL-1-38-1. Percent inhibition of PPAR stimulation by the respective agonists is indicated.

FIG. 3 shows an FP assay for PPAR binding. YL-1-38-1 was screened by FP, and its EC50 value was determined.

FIG. 4 shows a FPA for selective PPARδ binding. Three compounds binding to PPARδ were identified, but none were found to be selective by reporter assay.

FIG. 5 shows the docking of YL-1-38-1 to PPARγ LBD.

FIG. 6 shows the docking of BTB07995 to the PPARδ LBD. BTB07995 is positioned to attach to Cys249 of the PPARδ LBD. The trifluoromethyl-pyridyl group of BTB07995 was modeled to be conformationally flexible within the LBD and fit into either of the two arms (yellow and orange in the inset).

FIG. 7 shows PPAR reporter assays. Compounds were tested for their ability to inhibit activation of each PPAR in the presence of 1 μM agonist (WY14643, PPARα; GW7845, PPARγ; GW501516, PPARδ). Shown is the percent inhibition of PPAR stimulation by the respective agonists. HTS09910 and YL-1-38-1 indicated some PPARγ selectivity, and BTB07995 showed PPARδ selectivity at lower concentrations.

FIG. 8 shows PPAR reporter assay for compounds structurally related to BTB07995. Percent inhibition of PPAR stimulation by the respective selective agonists is indicated. Only BTB07995 had PPARδ selectivity. Some compounds were considered inactive.

FIG. 9 shows PPAR reporter assay for compounds structurally related to YL-1-38-1. Percent inhibition of PPAR stimulation by the respective selective agonists is indicated. Only YL-1-38-1 had PPARγ selectivity.

FIG. 10 shows structural analogs of YL-1-38-1 and HTS09910. Three analogs of YL-1-38-1 (A,B,C) and two analogs of HTS-00910 (A, B) are shown.

FIG. 11 shows the activity of BTB07995 in Gal4-mPPAR reporter assays in 293T cells. Each PPAR was assayed in the absence and presence of its specific ligand. Activity in the presence of 2.5-25 μM BTB07995 (A), and in the presence of 0.1-2.5 μM BTB07995 (B) after 24 hr.

FIG. 12 shows the BTB07995 analogs tested. The position of the sulfoxide is critical for PPARδ antagonism.

FIG. 13 shows the cytotoxicity of BTB07995 against mammary cell lines. Mouse mammary tumor cell lines MC, 437T, 105T and 34T were generated from primary DMBA-induced tumors in wild-type FVB, MMTV-Pax8PPARγ transgenic, Sca-1 null and Sca-1+/EGFP mice. Comma1D is an immortalized mammary epithelial cell line. Growth was determined in the absence and presence of PPARδ agonist GW501516 (GW) at 0, 2.5, 5, 10 and 25 μM BTB07995.

FIG. 14 shows a model of PPARδ in its antagonist conformation in complex with BTB07995. The model was developed based on the crystal structure of PPARα for folding predictions and PPARδ for side-chain predictions. BTB was docked, manually reoriented and further refined using stepwise Molecular Dynamics simulations for induced-fit model capability to consider displacement of residues. Shown are interactions between BTB07995 and Leu256, Thr289, His 323 and His 449.

FIG. 15 shows a comparison of BTB07995 bound to the three isoforms of PPAR. The AF-2 regions of the PPARs are colored in dark grey and BTB07995 is shown as a stick model with the carbon atoms in light grey. A, Binding to PPARα in the presence of antagonist GW6471 and a SMRT co-repressor peptide (PDB code: 1KKQ); the estimated inhibition constant (Kᵢ) of BTB07995 is 9.13 μM at 25° C. B, Binding to PPARα in the presence of agonist GW409544 and a SRC-1 activator peptide (PDB code: 1K7L), Kᵢ=1.20 μM. C, Binding to PPARγ in the presence of agonist GW4709 (PDB code: 2POB), Kᵢ=884 nM. D, Binding to PPARδ in the presence of agonist GW2331 (PDB code: 1Y0S), Kᵢ=627 nM. Residues interacting with BTB07995 are labeled.

FIG. 16 is a model of PPARγ in its antagonist conformation with compound Sd-107-10. Open conformation of helix-12 is shown as a ribbon model (magenta). (A) Ribbon model of Sd-107-10 interacting with PPARγ (ribbon model). (B) Detailed view of the interaction of Sd-107-10 (dark colored structure in the middle of the ribbon model) with the PPARγ pocket binding site. PPARγ residues interacting with Sd-107-10 are shown as a ball & stick model. Hydrogen bonds are shown as broken lines. The Sd-107-10 binding site is surrounded by hydrophobic and hydrophilic residues.

FIG. 17 shows a fluorescent Polarization Assay (FPA) of PPARγ with a fluorescent labeled co-repressor, NCoR peptide probe, and the YL-1-80 analogs. The binding activity is shown as a percentage of maximum and the minimum binding. YL-1-80 and YL-1-83 exhibited the best competition, and YL-1-83 was more selective for PPARγ in reporter assays (Table 1).

FIGS. 18A, 18B, 18C, 18D, and 18E show modeled interactions of YL-1-68-2 and YL-1-83 with PPARγ. A, Structure of YL-1-68-2. B-D, Modeled complex structure of YL-1-68-2 and PPARγ. B, Side-chain residues of PPARγ interacting with YL-1-68-2 are shown. C, AF-2 helix and YL-1-68-2 stretches into the three arms of the target binding site. D, The ligand binding pocket is shown in surface model colored with the electrostatic potential. E, Structure of YL-1-68-2. F, YL-1-83 binds to the ligand binding pocket similarly to YL-1-68-2.

### VII. DETAILED DESCRIPTION

### A. General

1. PPAR

The peroxisome proliferator-activated receptors (PPARs) are ligand-activated transcription factors of the nuclear receptor superfamily. They regulate glucose, lipid, and cholesterol metabolism in response to fatty acids and their derivatives. The PPAR subfamily contains three members known as PPARα, PPARβ/δ, and PPARγ (Willson, M. T. et al. J Med Chem 43:527-550). They are closely connected to cellular metabolism and cell differentiation. Three PPAR receptor subtypes with distinct tissue distributions, designated as PPARα, PPARγ and PPARβ/δ, have been identified. PPAR-α is expressed in certain tissues, including the liver, kidneys, heart, muscle and adipose. PPAR-γ, although transcribed by the same gene, exists in three forms. PPAR-γ 1 is expressed in virtually all tissues, including the heart, muscle, colon, kidneys, pancreas and the spleen. PPAR-γ 2 is expressed mainly in adipose tissue. PPAR-γ 3 is expressed in macrophages, the large intestine and white adipose tissue. PPAR-β/δ is expressed in a variety of tissues, including the brain, adipose and skin. The PPARs coordinate pathways involved in glucose and lipid homeostasis (Willson, M. T. et al. J Med Chem 43:527-550; Berger, J et al. Annu Rev Med 53:409-435, 2002). In addition, PPARγ and PPARβ/δ are involved in developmental and differentiation pathways and therefore play important roles in embryogenesis, inflammation and cancer (Zaveri, T. N. et al. Canc Biol Ther 8:1252-1261, 2009; Elikkottil, J. et al. Canc Biol Ther 8:1262-1264, 2009).

PPARs heterodimerize with retinoid X receptor (RXR) and bind to specific elements on the DNA of target genes called PPAR response elements. The binding of PPAR to its ligand then leads to an increase or decrease in gene expression. There are several known PPAR ligands such as, thiazolidinedione (TZD), fatty acids and the prostaglandin D2 metabolite 15d-PGJ2. The genes activated by PPAR-γ stimulate lipid uptake by fat cells.

There are three variants of PPARγ. Variants 1 and 3 have identical protein sequences. Variant 2 (protein id NP_{—}056953) has the same protein sequence as variants 1 and 3 but has the addition of 28 amino acids on the N-terminal end MGETLGDSPIDPESDSFTDTLSANISQE (SEQ ID NO:1). The majority of the nucleotide sequences are identical but there is variation at the N-terminal end of each variant. The first 169 bp of variant 1 are not present in variant 3. The first 196 bp of variant 3 are not present in variant 1. The final 1723 bp of variants 1 and 3 are identical. The final 1648 bp of variants 1 and 2 are identical. The first 244 bp of variant 1 are not present in variant 2. The first 172 bp of variant 2 are not present in variant 1.

### B. Compositions

Disclosed herein is a compound having the structure of:

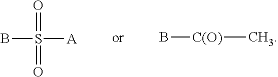

In some forms A can be:

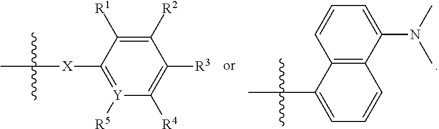

In some forms A can be

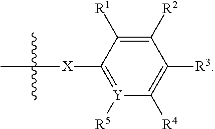

In some forms X can be absent or present, if present X can be —NH—. In some forms X can be absent.

In some forms Y can be C or N, if N R⁵ can be absent. In some forms Y can be C.

In some forms X can be absent and Y can be C. In some forms X can be absent and Y can be N and R⁵ can be absent.

In some forms R¹, R², R³, R⁴ and R⁵ can independently be hydrogen, C₁-C₃ alkyl, C₄-C₆ alkyl, C₁-C₆ alkyl, C₁-C₃ alkoxy, halo, C₁-C₃ haloalkyl, cyano or nitro, wherein at least one of R¹, R², R³, R⁴ and R⁵ is not hydrogen. In some forms at least two of R¹, R², R³, R⁴ and R⁵ are not hydrogen. In some forms at least three of R¹, R², R³, R⁴ and R⁵ are not hydrogen. In some forms at least four of R¹, R², R³, R⁴ and R⁵ are not hydrogen. In some forms R¹, R², R⁴ and R⁵ are hydrogen. In some forms R³ can be C₁-C₃ alkoxy, halo, C₁-C₃ haloalkyl, cyano or nitro. In some forms R³ can be methoxy, —CF₃, —CN or —Cl. In some forms R³ can be methoxy or —CF₃. In some forms R³ can be C₁-C₆ alkyl. In some forms R³ can be C₄ alkyl.

In some forms B can be:

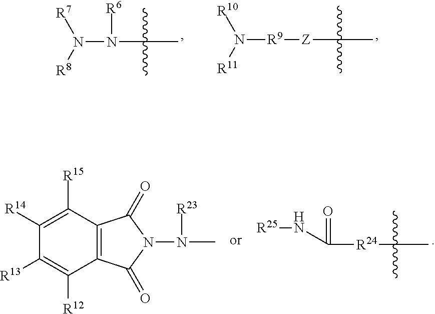

In some forms B can be

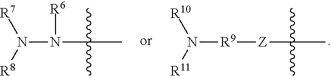

In some forms R⁶, R⁷ and R⁸ can independently be hydrogen, —C(O)—CH₂—R²²,

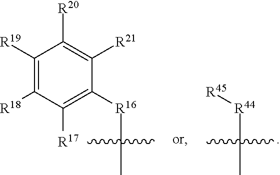

wherein at least one of R⁶, R⁷ and R⁸ is not hydrogen.

In some forms R⁶ and R⁷ are not hydrogen. In some forms R⁷ and R⁸ are not hydrogen. In some forms R⁶ is not hydrogen. In some forms R⁶, R⁷ and R⁸ are not hydrogen.

In some forms R¹⁶ can be —CH₂—, —CH₂CH₂—, —CH₂CH₂C(O)—, —CH₂C(O)—, or —C(O)—. In some forms R¹⁶ can be —C(O)— or —CH₂—. In some forms R¹⁶ can be —C(O)—.

In some forms R¹⁷, R¹⁸, R¹⁹, R²⁰ and R²¹ can independently be hydrogen, C₁-C₃ alkyl, C₄-C₆ alkyl, C₁-C₆ alkyl, C₁-C₃ alkoxy, halo, C₁-C₃ haloalkyl,

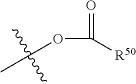

cyano or nitro, wherein at least one of R¹⁷, R¹⁸, R¹⁹, R²⁰ and R²¹ is not hydrogen. In some forms R¹⁹ can be methoxy, —CF₃, —CN, —NO₂,

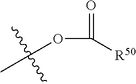

or —Cl. In some forms R¹⁹ can be methoxy,

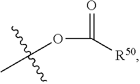

C₁-C₆ alkyl or —Cl.

In some forms R⁵⁰ can be H or C₁-C₆ alkyl. In some forms R⁵⁰ can be C₁ alkyl.

In some forms R⁴⁴ can be —CH₂—, —CH₂CH₂—, —CH₂CH₂C(O)—, —CH₂C(O)—, or —C(O)—. In some forms R⁴⁴ can be —C(O)— or —CH₂—. In some forms R⁴⁴ can be —C(O)—.

In some forms R⁴⁵ can be unsubstituted or substituted heteroaryl. In some forms R⁴⁵ can be a 6 membered substituted heteroaryl having 1-3 N atoms. In some form R⁴⁵ can be substituted pyridine. In some forms the substituted pyridine can be substituted with C₁-C₆ alkyl, hydrogen, C₁-C₃ alkoxy, halo, C₁-C₃ haloalkyl,

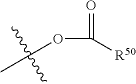

cyano or nitro. In some forms R⁴⁵ can have the structure

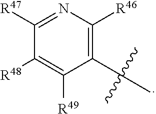

In some forms R⁴⁶, R⁴⁷, R⁴⁸, and R⁴⁹ can individually be H, hydroxyl, C₁-C₆ alkyl,

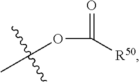

C₁-C₃ alkoxy, halo, C₁-C₃ haloalkyl, cyano or nitro alkyl, wherein at least one of R⁴⁶, R⁴⁷, R⁴⁸, and R⁴⁹ is not hydrogen. In some forms R⁴⁷ can be methoxy,

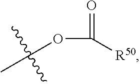

—CF₃, —CN, —NO₂ or —Cl. In some forms R⁴⁷ can be methoxy,

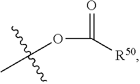

C₁-C₆ alkyl or —Cl.

In some forms R²² can be hydroxyl, halo, or hydrogen. In some forms R²² can be —Cl.

In some forms Z can absent or present, if present Z can be —N(H)—. In some forms Z can be absent.

In some forms R⁹ can be —CH₂—, —CH₂CH₂—, —CH₂CH₂C(O)—, —CH₂C(O)—, or —C(O)—. In some forms R⁹ can be —CH₂—, —CH₂CH₂— or —C(O)—. In some forms R⁹ can be —CH₂CH₂—.

In some forms R¹⁰ and R¹¹ can independently be hydrogen or

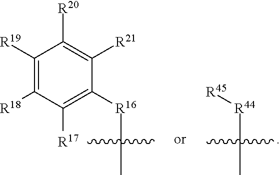

In some forms R²³ can be hydrogen or

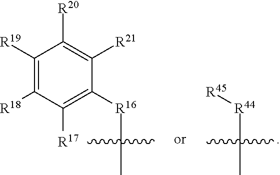

In some forms R²³ can be hydrogen.

In some forms R¹², R¹³, R¹⁴ and R¹⁵ can independently be hydrogen, C₁-C₃ alkyl, C₄-C₆ alkyl, C₁-C₆ alkyl,

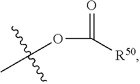

C₁-C₃ alkoxy, halo, C₁-C₃ haloalkyl, cyano or nitro, wherein at least one of R¹², R¹³, R¹⁴ and R¹⁵ is not hydrogen. In some forms R¹² and R¹⁵ can be hydrogen. In some form R¹³ and R¹⁴ can independently be methoxy or halo. In some forms R¹³ and R¹⁴ can be —Cl.

In some forms R²⁴ can be —CH₂—, —CH₂CH₂—, —CH₂CH₂CH₂— or —CH₂CH₂CH₂CH₂—. In some forms R²⁴ can be —CH₂CH₂—.

In some forms R²⁵ can be

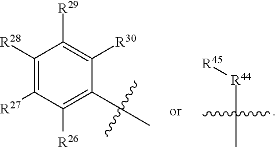

In some forms R²⁶, R²⁷, R²⁸, R²⁹ and R³⁰ are independently hydrogen, C₁-C₃ alkyl, C₁-C_{3 i} alkoxy, halo, C₁-C₃ haloalkyl, cyano or nitro, wherein at least one of R²⁶, R²⁷, R²⁸, R²⁹ and R³⁰ is not hydrogen. In some forms R²⁸ can be methoxy, —CN, —CF₃ or —Cl.

In some forms the compound is not

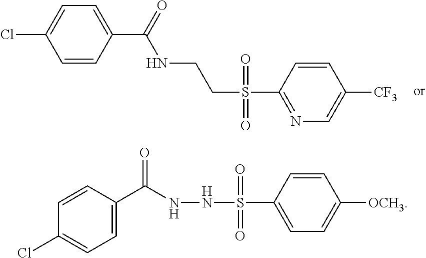

In some forms R⁶ and R⁷ can be

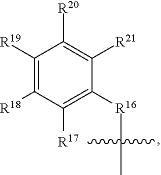

R⁸ can be H, wherein R¹⁶ can be C(O), R¹⁷, R¹⁸, R²⁰ and R²¹ can be H and R¹⁹ can be hydroxyl, —Cl or C₁-C₆ alkyl.

In some forms the compound

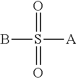

and B—C(O)—CH₃ can have the structure:

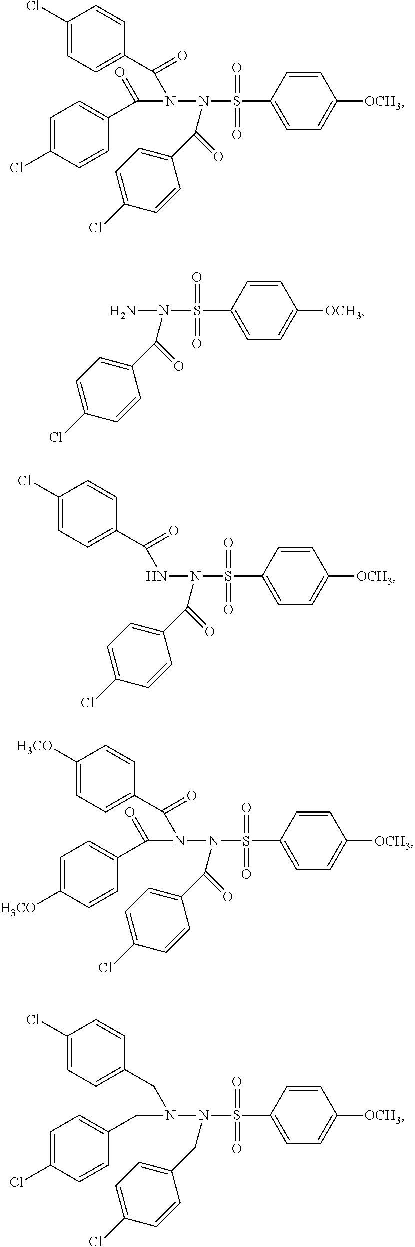

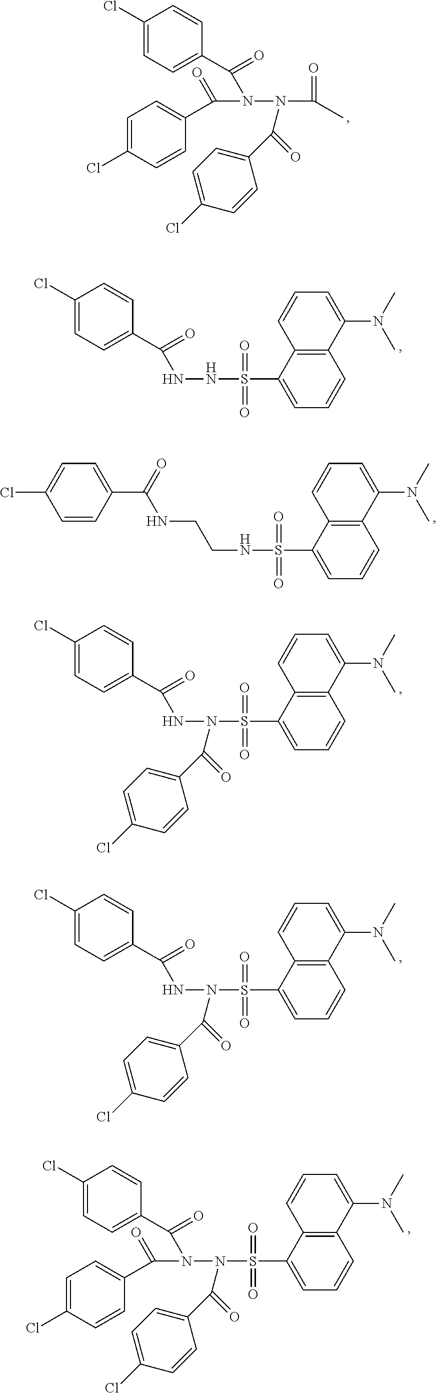 [SMILES: CC(=O)N(C(=O)c1ccc(Cl)cc1)N(C(=O)c1ccc(Cl)cc1)C(=O)c1ccc(Cl)cc1 ; CN(C)c1cccc2c(S(=O)(=O)NNC(=O)c3ccc(Cl)cc3)cccc12 ; CN(C)c1cccc2c(S(=O)(=O)NCCNC(=O)c3ccc(Cl)cc3)cccc12 ; CN(C)c1cccc2c(S(=O)(=O)N(NC(=O)c3ccc(Cl)cc3)C(=O)c3ccc(Cl)cc3)cccc12 ; CN(C)c1cccc2c(S(=O)(=O)N(NC(=O)c3ccc(Cl)cc3)C(=O)c3ccc(Cl)cc3)cccc12 ; CN(C)c1cccc2c(S(=O)(=O)N(C(=O)c3ccc(Cl)cc3)N(C(=O)c3ccc(Cl)cc3)C(=O)c3ccc(Cl)cc3)cccc12]

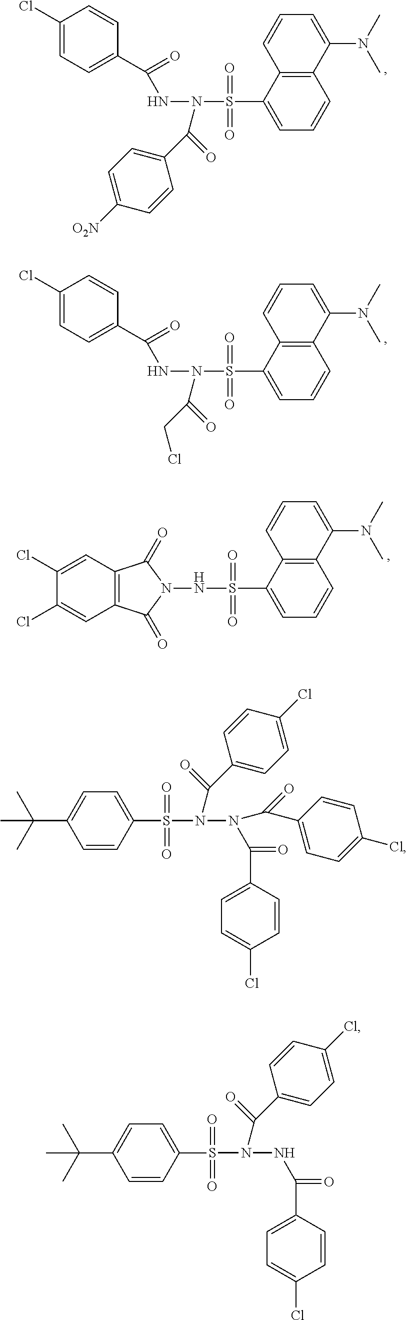

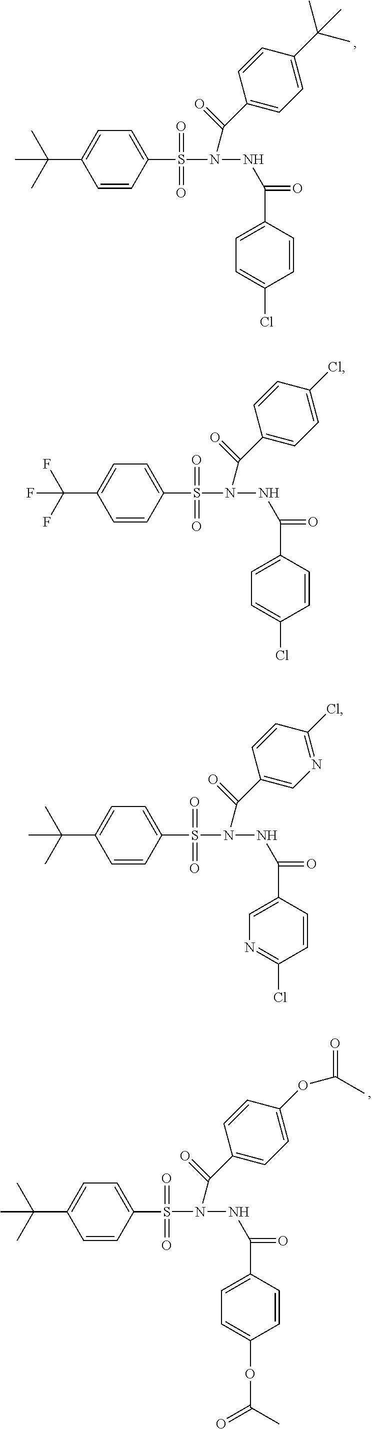

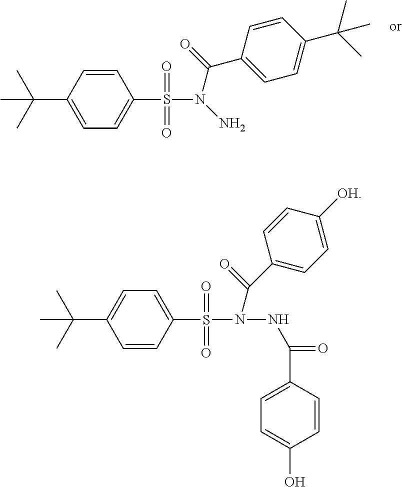

Also disclosed herein are compounds having the structure of:

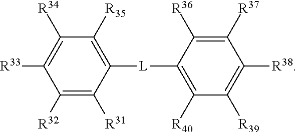

In some forms L can be —C(O)CHCH—, —C(O)(CH₂)₁₋₃—, —C(O)(CHCH)₂—, —(CHCH)₁₋₂ or —(CH₂)₁₋₄—. In some forms L can be —C(O)CHCH.

In some forms R³¹, R³², R³³, R³⁴, R³⁵, R³⁶, R³⁷, R³⁸, R³⁹ or R⁴⁰ can independently be hydrogen, —B(OH)₂, C₁-C₃ alkyl, C₁-C₃ alkoxy, halo, C₁-C₃ haloalkyl, cyano or nitro, wherein at least four of R³¹, R³², R³³, R³⁴, R³⁵, R³⁶, R³⁷, R³⁸, R³⁹ or R⁴⁰ are not hydrogen. In some forms at least five of R³¹, R³², R³³, R³⁴, R³⁵, R³⁶, R³⁷, R³⁸, R³⁹ or R⁴⁰ are not hydrogen. In some forms R³¹, R³⁵, R³⁶, R³⁹ or R⁴⁰ can be hydrogen. In some forms R³², R³³, R³⁴, R³⁷ and R³⁸ can independently be methoxy, halo or —B(OH)₂. In some forms R³⁷ can be —B(OH)₂.

In some forms structure

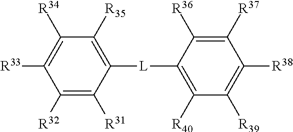

can have the structure

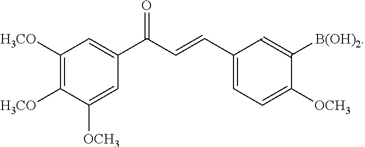 [SMILES: COc1ccc(/C=C/C(=O)c2cc(OC)c(OC)c(OC)c2)cc1B(O)O]

Also disclosed is a compound having the structure of:

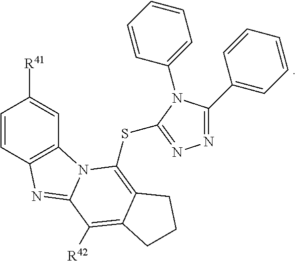

In some forms R⁴¹ can be hydrogen, hydroxyl, halo, C₁-C₃ alkyl, C₁-C₃ alkoxy, C₁-C₃ haloalkyl, nitro, cyano or —B(OH)₂.

In some forms R⁴² can be hydrogen hydroxyl, halo, C₁-C₃ alkyl, C₁-C₃ alkoxy, C₁-C₃ haloalkyl, nitro, cyano, —B(OH)₂ or —C(O)—R⁴³.

In some forms R⁴³ can be C₁-C₃ alkyl or hydrogen.

In some forms R⁴¹ and R⁴² are not both hydrogen.

In some forms R⁴¹ is not hydrogen if R⁴² can be cyano.

Also disclosed is a compound having the structure of:

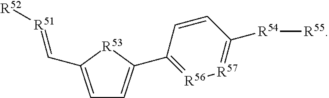

In some forms R⁵¹ can be a heterocyclic structure having two substituents selected from ═O and ═S. In some forms R⁵¹ can be a 5 membered heterocyclic structure having two substituents selected from ═O and ═S. In some forms R⁵¹ can be pyrazolidine-3,5,dione, 2-thioxothiazolidin-4-1, 2-thioxooxazolidin-4-1, thiazolidine-2,4-dione or 5-thioxopyrazolidin-3-1.

In some forms R⁵² can be substituted or unsubstituted aryl, substituted or unsubstituted heteroaryl, substituted or unsubstituted cycloalkyl, substituted or unsubstituted heterocyclyl, 1-methylcyclopropanecarboxylate C₁-C₆ alkyl,

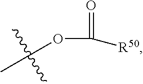

C₁-C₃ alkoxy, halo, C₁-C₃ haloalkyl, cyano or nitro. In some forms R⁵² can be phenyl, ethyl, butyl, cyclohexyl, biphenyl, phenoxybenzyl propyl 1-methylcyclopropanecarboxylate or halogenated benzene. In some forms R⁵² can be fluoro substituted benzene.

In some forms R⁵³ can be O, S or NH. In some forms R⁵³ can be O.

In some forms R⁵⁶ can be CH and R⁵⁷ can be CH. In some forms R⁵⁶ can be N and R⁵⁷ can be CH. In some forms R⁵⁶ can be CH and R⁵⁷ can be N.

In some forms R⁵⁴ can be —SO₂—, —NH—, —S(O)₂NH—, —NHCH₂—, —NHCH₂CH₂—, —NHCH₂CH₂CH₂—, —NHCOO—, —SO₂NHCOO— or —SO₂NHC(O)—. In some forms R⁵⁴ can be —SO₂— or —S(O)₂NH—.

In some forms R⁵⁵ can be H, C₁-C₃ alkyl, heteroaryl, heterocyclyl, aryl or cycloalkyl. In some forms R⁵⁵ can be H, C₁-C₃ alkyl, phenyl, pyrrole imidazole, oxazole, thiazole or triazole.

In some form the compound can have the structure:

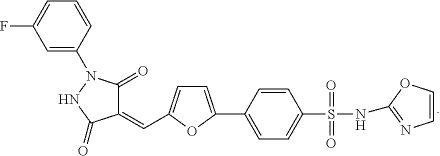 [SMILES: O=C1NN(c2cccc(F)c2)C(=O)/C1=C\c1ccc(-c2ccc(S(=O)(=O)Nc3ncco3)cc2)o1]

1. Synthesis

YL-1-38-1 was synthesized by simple acetylation reaction (Scheme 1), at the same time three other interesting analogs were also obtained.

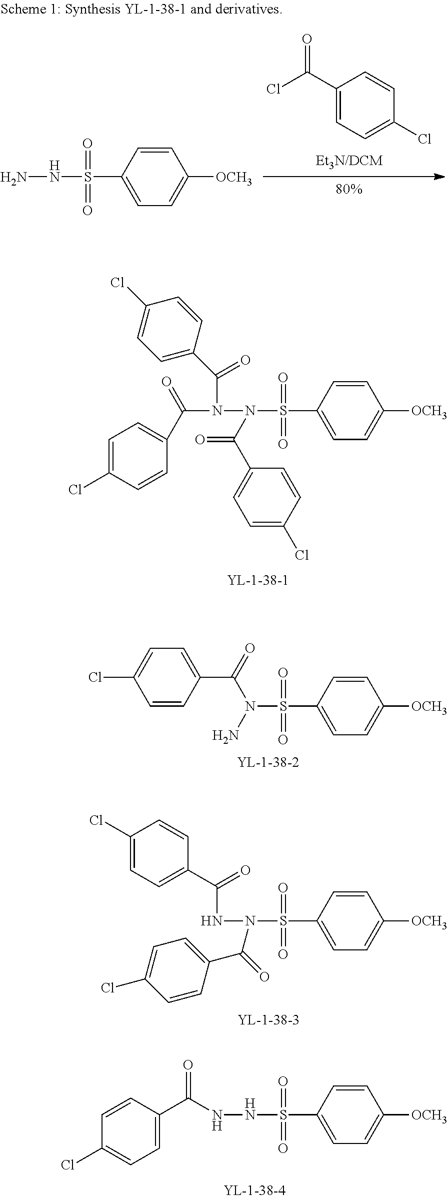 [SMILES: O=C(Cl)c1ccc(Cl)cc1 ; COc1ccc(S(=O)(=O)NN)cc1 ; COc1ccc(S(=O)(=O)N(C(=O)c2ccc(Cl)cc2)N(C(=O)c2ccc(Cl)cc2)C(=O)c2ccc(Cl)cc2)cc1 ; COc1ccc(S(=O)(=O)N(N)C(=O)c2ccc(Cl)cc2)cc1 ; COc1ccc(S(=O)(=O)N(NC(=O)c2ccc(Cl)cc2)C(=O)c2ccc(Cl)cc2)cc1 ; COc1ccc(S(=O)(=O)NNC(=O)c2ccc(Cl)cc2)cc1]

Synthesis procedure for YL-1-38-1: To the mixture of 4-Methoxybenzene-sulfonyl hydrazide (1 g, 4.94 mmol) and triethyl amine (1.4 ml, 10 mmol) in dichloromethylene (40 ml), 4-chlorobenzoyl chloride (0.63 ml, 4.94 mmol) was added dropwisely at −20° C.-10° C. under nitrogen. The reaction mixture was stirred for another 30 mins after adding. The saturated aqueous solution of NH₄Cl (5 ml) was added, then ethyl acetate (100 ml) was added. The organic phase was washed by water (3×20 mL) and Brine (3×20 mL), then dried by MgSO₄ for 10 mins. Then filtered and the filtration was concentrated under vacuum, the residue was purified by column chromatography to give 200 mg of YL-1-38-1, 110 mg of YL-1-38-2, 30 mg YL-1-38-3 and 20 mg YL-1-38-4. Yield was 64.5% based on 4-chlorobenzoyl chloride.

2. General Compositions

i. Pharmaceutical Carriers and Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue. (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue. (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed. (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

ii. Therapeutic Uses

Effective dosages and schedules for administering the compositions can be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing a cancer, such as prostate cancer, the efficacy of the therapeutic antibody can be assessed in various ways well known to the skilled practitioner

The compositions that inhibit disclosed ER and cancer, such as breast cancer, interactions disclosed herein can be administered as a therapy or prophylactically to patients or subjects who are at risk for the cancer or breast cancer.

3. Compositions Identified by Screening with Disclosed Compositions/Combinatorial Chemistry

i. Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions disclosed herein, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, disclosed ER and Compounds 1-6s, are also disclosed. Thus, the products produced using the combinatorial or screening approaches that involve the disclosed compositions, such as, disclosed ERs and Compounds 1-6, are also considered herein disclosed.

It is understood that the disclosed methods for identifying molecules that inhibit the interactions between, for example, disclosed ERs and Compounds 1-6 can be performed using high through put means. For example, putative inhibitors can be identified using Fluorescence Resonance Energy Transfer (FRET) to quickly identify interactions. The underlying theory of the techniques is that when two molecules are close in space, i.e., interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514). Libraries using the disclosed compounds, such as Compounds 1-6 can be made.

As used herein combinatorial methods and libraries included traditional screening methods and libraries as well as methods and libraries used in interactive processes.

ii. Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, disclosed ERs and Compounds 1-6, are also disclosed. Thus, the products produced using the molecular modeling approaches that involve the disclosed compositions, such as, disclosed ERs and Compounds 1-6s, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable determination of how a new compound will link to the target molecule and allow experimental manipulation of the structures of the compound and target molecule to perfect binding specificity. Modeling of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### C. Methods

Also disclosed herein are methods of inhibiting peroxisome proliferator-activating receptors (PPARs) comprising administering a composition comprising a compound having the structure:

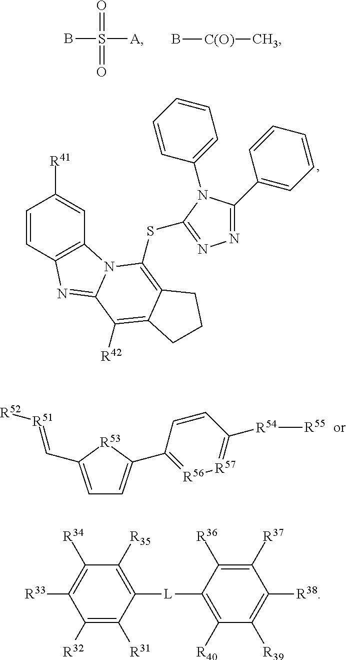

Also disclosed herein are methods of treating cancer comprising administering a composition comprising a compound having the structure:

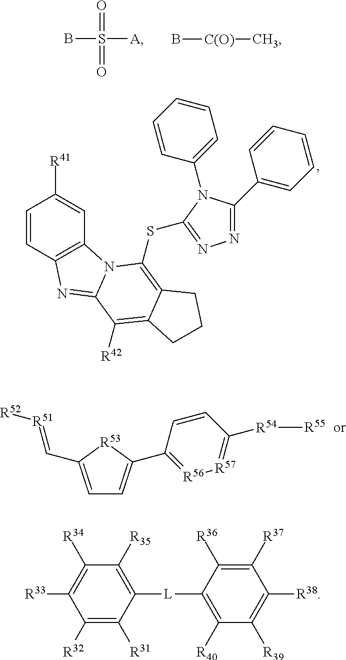

Also disclosed herein are methods of treating metabolic disorders comprising administering a composition comprising a compound having the structure:

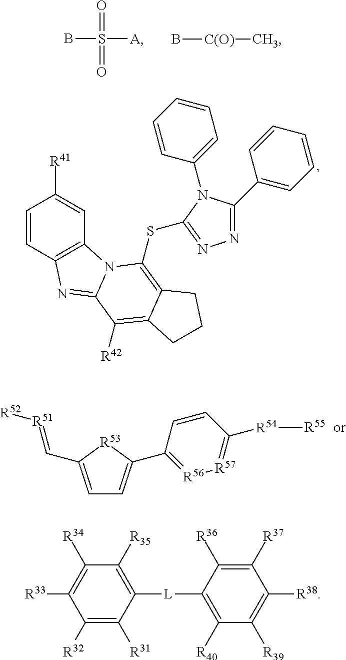

Also disclosed herein are methods of preventing or treating a PPAR-mediated disease or condition comprising administering a therapeutically effective amount of a composition comprising a compound having the structure:

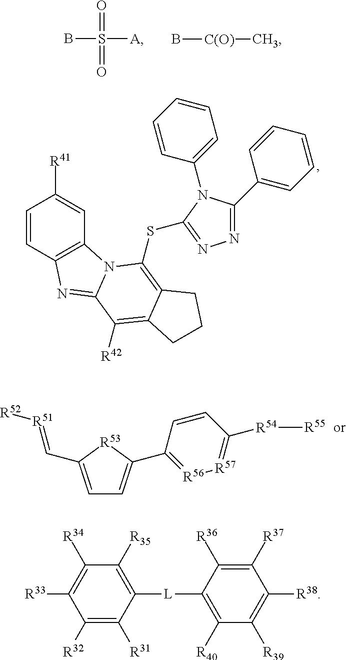

In some forms, the disclosed compounds can be a pharmaceutically acceptable salt, prodrug, clathrate, tautomer or solvate thereof.

In some forms A can be:

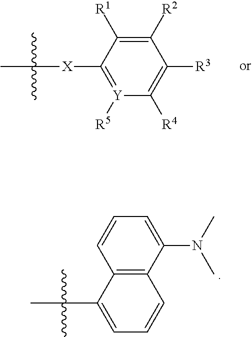

In some forms A can be

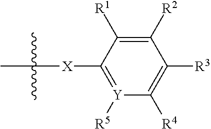

In some forms X can be absent or present, if present X can be —NH—. In some forms X can be absent.

In some forms Y can be C or N, if N R⁵ can be absent. In some forms Y can be C.

In some forms X can be absent and Y can be C. In some forms X can be absent and Y can be N and R⁵ can be absent.

In some forms R¹, R², R³, R⁴ and R⁵ can independently be hydrogen, C₁-C₃ alkyl, C₄-C₆ alkyl, C₁-C₆ alkyl, C₁-C₃ alkoxy, halo, C₁-C₃ haloalkyl, cyano or nitro, wherein at least one of R¹, R², R³, R⁴ and R⁵ is not hydrogen. In some forms at least two of R¹, R², R³, R⁴ and R⁵ are not hydrogen. In some forms at least three of R¹, R², R³, R⁴ and R⁵ are not hydrogen. In some forms at least four of R¹, R², R³, R⁴ and R⁵ are not hydrogen. In some forms R¹, R², R⁴ and R⁵ are hydrogen. In some forms R³ can be C₁-C₃ alkoxy, halo, C₁-C₃ haloalkyl, cyano or nitro. In some forms R³ can be methoxy, —CF₃, —CN or —Cl. In some forms R³ can be methoxy or —CF₃. In some forms R³ can be C₁-C₆ alkyl. In some forms R³ can be C₄ alkyl.

In some forms B can be:

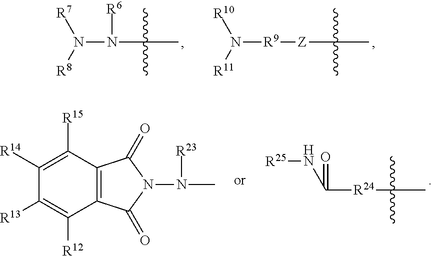

In some forms B can be

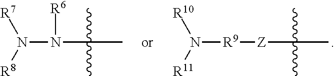

In some forms R⁶, R⁷ and R⁸ can independently be hydrogen, —C(O)—CH₂—R²²,

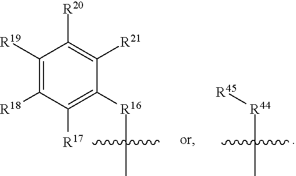

wherein at least one of R⁶, R⁷ and R⁸ is not hydrogen.

In some forms R⁶ and R⁷ are not hydrogen. In some forms R⁷ and R⁸ are not hydrogen. In some forms R⁶ is not hydrogen. In some forms R⁶, R⁷ and R⁸ are not hydrogen.

In some forms R¹⁶ can be —CH₂—, —CH₂CH₂—, —CH₂CH₂C(O)—, —CH₂C(O)—, or —C(O)—. In some forms R¹⁶ can be —C(O)— or —CH₂—. In some forms R¹⁶ can be —C(O)—.

In some forms R¹⁷, R¹⁸, R¹⁹, R²⁰ and R²¹ can independently be hydrogen, C₁-C₃ alkyl, C₄-C₆ alkyl, C₁-C₆ alkyl, C₁-C₃ alkoxy, halo, C₁-C₃ haloalkyl,

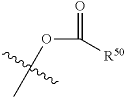

cyano or nitro, wherein at least one of R¹⁷, R¹⁸, R¹⁹, R²⁰ and R²¹ is not hydrogen. In some forms R¹⁹ can be methoxy, —CF₃, —CN, —NO₂,

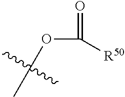

or —Cl. In some forms R¹⁹ can be methoxy,

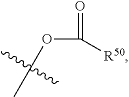

C₁-C₆ alkyl or —Cl.

In some forms R⁵⁰ can be H or C₁-C₆ alkyl. In some forms R⁵⁰ can be C₁ alkyl.

In some forms R⁴⁴ can be —CH₂—, —CH₂CH₂—, —CH₂CH₂C(O)—, —CH₂C(O)—, or —C(O)—. In some forms R⁴⁴ can be —C(O)— or —CH₂—. In some forms R⁴⁴ can be —C(O)—.

In some forms R⁴⁵ can be unsubstituted or substituted heteroaryl. In some forms R⁴⁵ can be a 6 membered substituted heteroaryl having 1-3 N atoms. In some form R⁴⁵ can be substituted pyridine. In some forms the substituted pyridine can be substituted with C₁-C₆ alkyl, hydrogen, C₁-C₃ alkoxy, halo, C₁-C₃ haloalkyl,

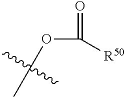

cyano or nitro. In some forms R⁴⁵ can have the structure

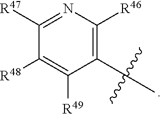

In some forms R⁴⁶, R⁴⁷, R⁴⁸, and R⁴⁹ can individually be H, hydroxyl, C₁-C₆ alkyl,

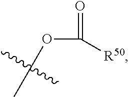

C₁-C₃ alkoxy, halo, C₁-C₃ haloalkyl, cyano or nitro alkyl, wherein at least one of R⁴⁶, R⁴⁷, R⁴⁸, and R⁴⁹ is not hydrogen. In some forms R⁴⁷ can be methoxy,

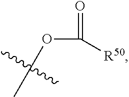

—CF₃, —CN, —NO₂ or —Cl. In some forms R⁴⁷ can be methoxy,

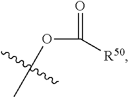

C₁-C₆ alkyl or —Cl.

In some forms R²² can be hydroxyl, halo, or hydrogen. In some forms R²² can be —Cl.

In some forms Z can absent or present, if present Z can be —N(H)—. In some forms Z can be absent.

In some forms R⁹ can be —CH₂—, —CH₂CH₂—, —CH₂CH₂C(O)—, —CH₂C(O)—, or —C(O)—. In some forms R⁹ can be —CH₂—, —CH₂CH₂— or —C(O)—. In some forms R⁹ can be —CH₂CH₂—.

In some forms R¹⁰ and R¹¹ can independently be hydrogen or

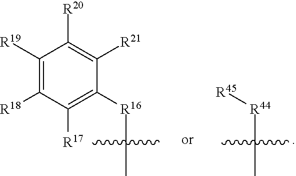

In some forms R²³ can be hydrogen or

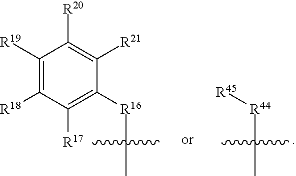

In some forms R²³ can be hydrogen.

In some forms R¹², R¹³, R¹⁴ and R¹⁵ can independently be hydrogen, C₁-C₃ alkyl, C₄-C₆ alkyl, C₁-C₆ alkyl,

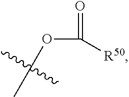

C₁-C₃ alkoxy, halo, C₁-C₃ haloalkyl, cyano or nitro, wherein at least one of R¹², R¹³, R¹⁴ and R¹⁵ is not hydrogen. In some forms R¹² and R¹⁵ can be hydrogen. In some form R¹³ and R¹⁴ can independently be methoxy or halo. In some forms R¹³ and R¹⁴ can be —Cl.

In some forms R²⁴ can be —CH₂—, —CH₂CH₂—, —CH₂CH₂CH₂— or —CH₂CH₂CH₂CH₂—. In some forms R²⁴ can be —CH₂CH₂—.

In some forms R²⁵ can be

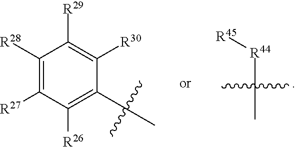

In some forms R²⁶, R²⁷, R²⁸, R²⁹ and R³⁰ are independently hydrogen, C₁-C₃ alkyl, C₁-C_{3 i} alkoxy, halo, C₁-C₃ haloalkyl, cyano or nitro, wherein at least one of R²⁶, R²⁷, R²⁸, R²⁹ and R³⁰ is not hydrogen. In some forms R²⁸ can be methoxy, —CN, —CF₃ or —Cl.

In some forms L can be —C(O)CHCH—, —C(O)(CH₂)₁₋₃—, —C(O)(CHCH)₂—, —(CHCH)₁₋₂ or —(CH₂)₁₋₄—. In some forms L can be —C(O)CHCH.

In some forms R³¹, R³², R³³, R³⁴, R³⁵, R³⁶, R³⁷, R³⁸, R³⁹ or R⁴⁰ can independently be hydrogen, —B(OH)₂, C₁-C₃ alkyl, C₁-C₃ alkoxy, halo, C₁-C₃ haloalkyl, cyano or nitro, wherein at least four of R³¹, R³², R³³, R³⁴, R³⁵, R³⁶, R³⁷, R³⁸, R³⁹ or R⁴⁰ are not hydrogen. In some forms at least five of R³¹, R³², R³³, R³⁴, R³⁵, R³⁶, R³⁷, R³⁸, R³⁹ or R⁴⁰ are not hydrogen. In some forms R³¹, R³⁵, R³⁶, R³⁹ or R⁴⁰ can be hydrogen. In some forms R³², R³³, R³⁴, R³⁷ and R³⁸ can independently be methoxy, halo or —B(OH)₂. In some forms R³⁷ can be —B(OH)₂.

In some forms R⁴¹ can be hydrogen, hydroxyl, halo, C₁-C₃ alkyl, C₁-C₃ alkoxy, C₁-C₃ haloalkyl, nitro, cyano or —B(OH)₂.

In some forms R⁴² can be hydrogen hydroxyl, halo, C₁-C₃ alkyl, C₁-C₃ alkoxy, C₁-C₃ haloalkyl, nitro, cyano, —B(OH)₂ or —C(O)—R⁴³.

In some forms R⁴³ can be C₁-C₃ alkyl or hydrogen.

In some forms R⁴¹ and R⁴² are not both hydrogen.

In some forms R⁴¹ is not hydrogen if R⁴² can be cyano.

In some forms R⁵¹ can be a heterocyclic structure having two substituents selected from ═O and ═S. In some forms R⁵¹ can be a 5 membered heterocyclic structure having two substituents selected from ═O and ═S. In some forms R⁵¹ can be pyrazolidine-3,5,dione, 2-thioxothiazolidin-4-1, 2-thioxooxazolidin-4-1, thiazolidine-2,4-dione or 5-thioxopyrazolidin-3-1.

In some forms R⁵² can be substituted or unsubstituted aryl, substituted or unsubstituted heteroaryl, substituted or unsubstituted cycloalkyl, substituted or unsubstituted heterocyclyl, 1-methylcyclopropanecarboxylate C₁-C₆ alkyl,

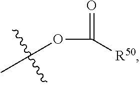

C₁-C₃ alkoxy, halo, C₁-C₃ haloalkyl, cyano or nitro. In some forms R⁵² can be phenyl, ethyl, butyl, cyclohexyl, biphenyl, phenoxybenzyl propyl 1-methylcyclopropanecarboxylate or halogenated benzene. In some forms R⁵² can be fluoro substituted benzene.

In some forms R⁵³ can be O, S or NH. In some forms R⁵³ can be O.

In some forms R⁵⁶ can be CH and R⁵⁷ can be CH. In some forms R⁵⁶ can be N and R⁵⁷ can be CH. In some forms R⁵⁶ can be CH and R⁵⁷ can be N.

In some forms R⁵⁴ can be —SO₂—, —NH—, —S(O)₂NH—, —NHCH₂—, —NHCH₂CH₂—, —NHCH₂CH₂CH₂—, —NHCOO—, —SO₂NHCOO— or —SO₂NHC(O)—. In some forms R⁵⁴ can be —SO₂— or —S(O)₂NH—.

In some forms R⁵⁵ can be H, C₁-C₃ alkyl, heteroaryl, heterocyclyl, aryl or cycloalkyl. In some forms R⁵⁵ can be H, C₁-C₃ alkyl, phenyl, pyrrole imidazole, oxazole, thiazole or triazole.

In some forms structures

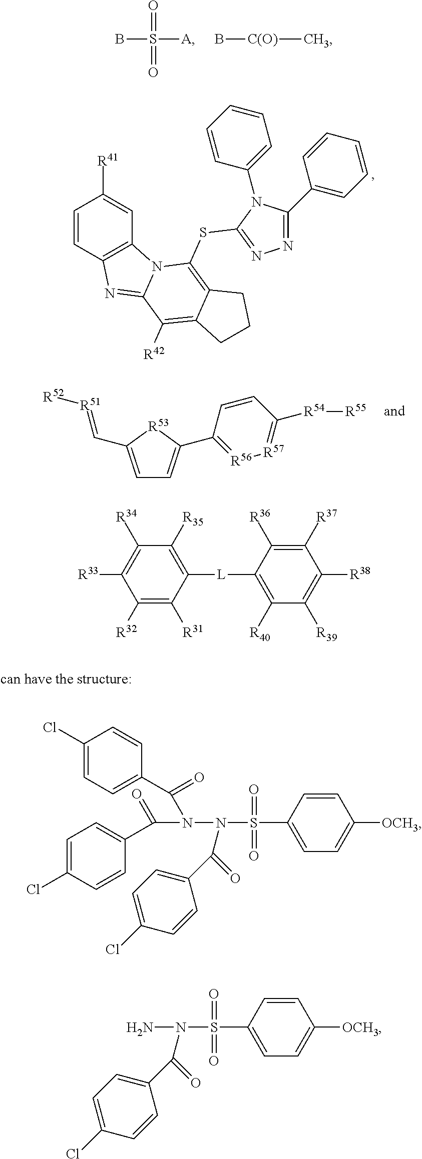

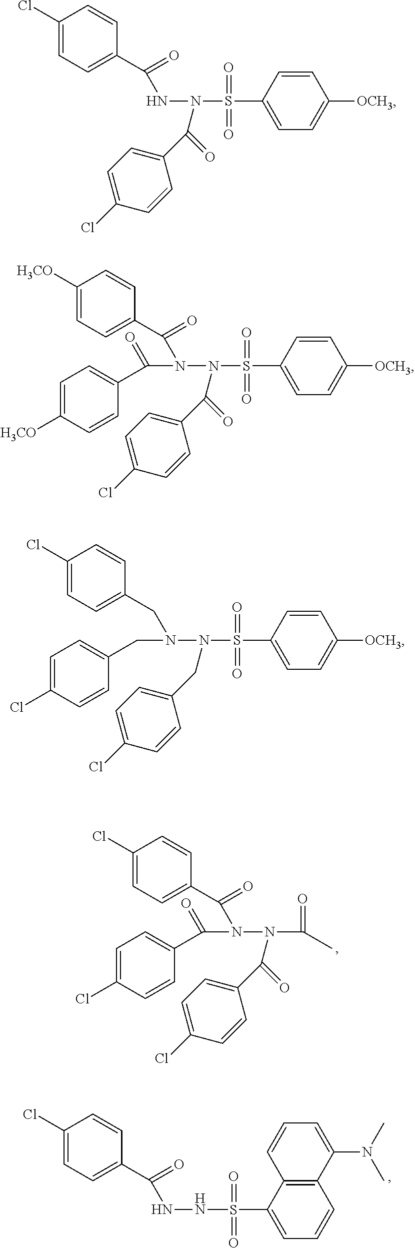

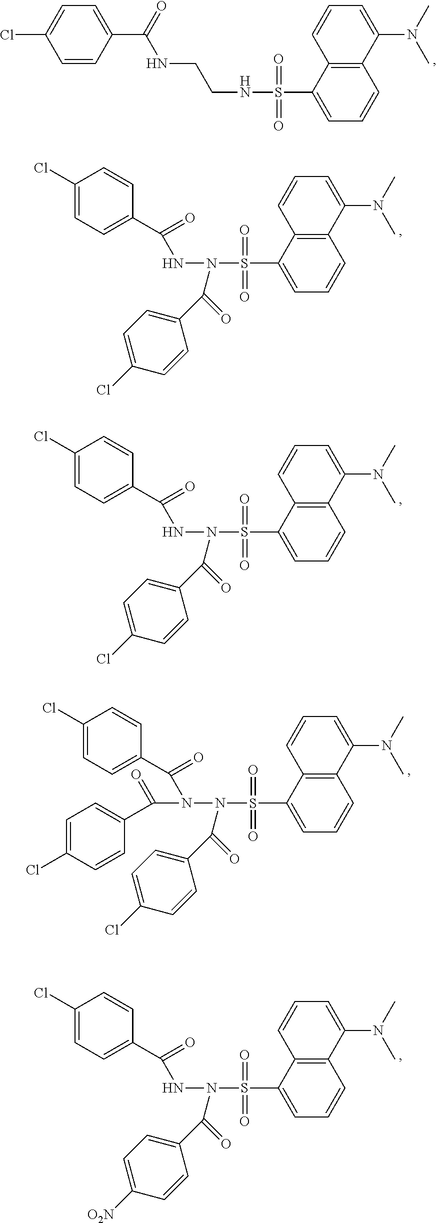 [SMILES: CN(C)c1cccc2c(S(=O)(=O)NCCNC(=O)c3ccc(Cl)cc3)cccc12 ; CN(C)c1cccc2c(S(=O)(=O)N(NC(=O)c3ccc(Cl)cc3)C(=O)c3ccc(Cl)cc3)cccc12 ; CN(C)c1cccc2c(S(=O)(=O)N(NC(=O)c3ccc(Cl)cc3)C(=O)c3ccc(Cl)cc3)cccc12 ; CN(C)c1cccc2c(S(=O)(=O)N(C(=O)c3ccc(Cl)cc3)N(C(=O)c3ccc(Cl)cc3)C(=O)c3ccc(Cl)cc3)cccc12 ; CN(C)c1cccc2c(S(=O)(=O)N(NC(=O)c3ccc(Cl)cc3)C(=O)c3ccc([N+](=O)[O-])cc3)cccc12]

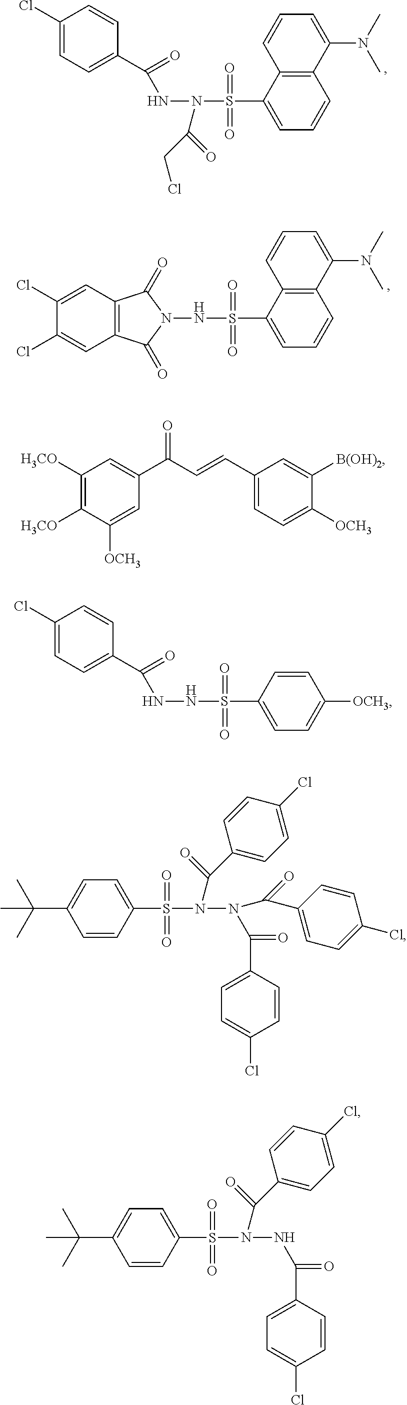

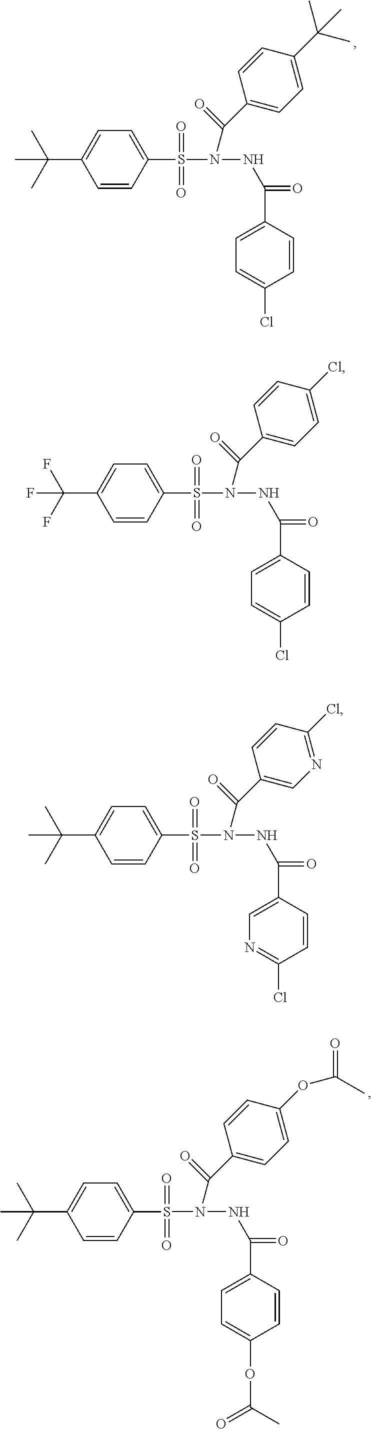

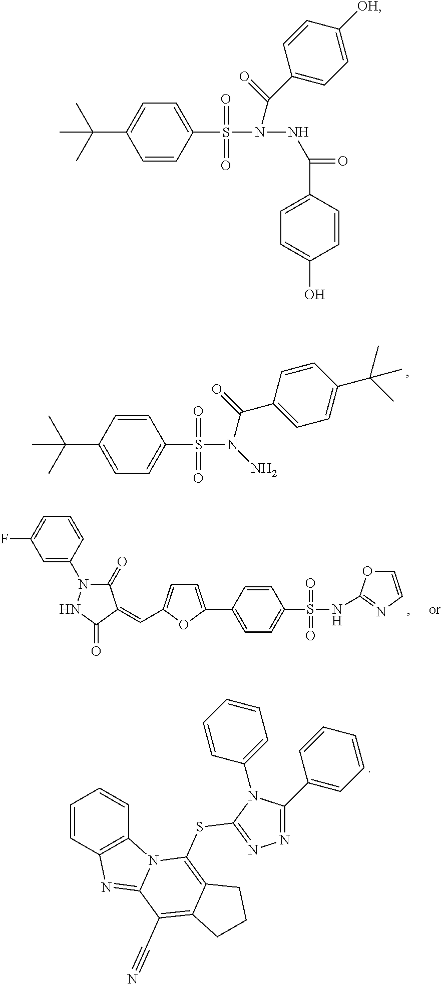

In some forms, a therapeutically effective amount of the composition can be administered.

1. Inhibiting PPAR

The compositions disclosed in the methods of inhibiting PPARs can be PPAR antagonists.

In some forms, the disclosed methods of inhibiting PPARs can inhibit PPARγ, PPARδ, or PPARα.

2. Treating Cancer

The compositions disclosed in the methods of treating cancer can be PPAR antagonists. The PPAR antagonists can be PPARγ, PPARδ, or PPARα antagonists.

In some forms of the disclosed methods of treating cancer, the composition can induce estrogen receptor alpha (ERα) expression in cancer cells. In some forms, the cancer cells can be ERα negative. In some forms, the cancer cells can be ERα positive but levels of ERα are too low for the cancer cells to be ERα dependent. In some forms, the induction of ERα expression results in ERα dependent cancer cells.

In some forms, the ERα dependent cancer cells are responsive to anti-estrogen therapy. In some forms, the disclosed methods of treating cancer can further comprise administering an anti-estrogen therapy. The anti-estrogen therapy can be effective for treating ERα dependent cancers. In some forms, the level of ERα expression is sufficient for the cancer cells to become dependent on ERα.

In some forms of the disclosed methods of treating cancer, a subject can be assayed for cancer or a risk of cancer. In some forms, a subject can be at risk of having cancer. In some forms, a subject can have cancer.

In some forms, the cancer is breast cancer. In some forms, the cancer is ERα positive.

3. Treating Metabolic Disorders

In some forms of the methods of treating metabolic disorders, the metabolic disorder is dislipidemia or diabetes. In some forms the diabetes is Type II diabetes. The metabolic disorders can be any disorder or disease that affects the process the body uses to get or make energy from food. Examples of metabolic disorders include, but are not limited to, Lesch-Nyhan Syndrome, mitochondrial disorders, Pompe Disease, Glycogen Storage Diseases, Amyloidosis, Tay-Sachs, Lysosomal disorders, Wilson's disease, Leukodystrophies, Phenylketonuria, Calcium disorders, Paget's disease, Mucopolysaccharidoses, and Gaucher disease.

In some forms of the disclosed methods of treating metabolic disorders, a subject can be assayed for metabolic disorders or a risk of metabolic disorders. In some forms, a subject can be at risk of having a metabolic disorder. In some forms, a subject can have a metabolic disorder. In some forms, the metabolic disorder is genetic.

4. Preventing/Treating PPAR-Mediated Disease

In some forms of the methods of preventing or treating PPAR-mediated disease or condition, the PPAR-mediated disease or condition can be a PPARγ-mediated disease or condition.

In some forms of the disclosed methods, the disease or condition can be selected from the group consisting of diabetes, obesity, metabolic syndrome, impaired glucose tolerance, syndrome X, and cardiovascular disease. In some forms, the disease or condition can be selected from the group consisting of diabetes and cardiovascular disease.

In some forms, the PPAR-mediated disease or PPARγ-mediated disease can be due to increased or decreased activity of PPAR or PPARγ. In some forms PPAR or PPARγ expression levels are higher than compared to a standard or control. The standard or control can be expression levels of PPAR or PPARγ in a normal or healthy individual.

### D. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for administering compositions, such as those disclosed herein, the kit comprising a composition and a means for administering the composition to a subject. The kits also can contain protocols for administering the compositions.

### E. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising cells, compounds, and instruments for detecting binding.

### F. Data Structures and Computer Control

Disclosed are data structures used in, generated by, or generated from, the disclosed method. Data structures generally are any form of data, information, and/or objects collected, organized, stored, and/or embodied in a composition or medium.

The disclosed method, or any part thereof or preparation therefore, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### G. Uses

The disclosed compositions can be used in a variety of ways as research tools. Other uses are disclosed, apparent from the disclosure, and/or will be understood by those in the art.

### H. Definitions

Various embodiments of the disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

1. A

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like.

2. Abbreviations

Abbreviations, which are well known to one of ordinary skill in the art, can be used (e.g., “h” or “hr” for hour or hours, “g” or “gm” for gram(s), “mL” for milliliters, and “rt” for room temperature, “nm” for nanometers, “M” for molar, and like abbreviations).

3. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

4. Anti-Estrogen Therapy

The term “anti-estrogen therapy” refers to a treatment with a composition that blocks or interferes with estrogen. In one example, anti-estrogen therapy can be an antibody that prevents estrogen from binding to ERα.

5. Clathrate

A compound for use in the and with the disclosed compounds, compositions, and methods can form a complex such as a “clathrate”, a drug-host inclusion complex, wherein, in contrast to solvates, the drug and host are present in stoichiometric or non-stoichiometric amounts. A compound used herein can also contain two or more organic and/or inorganic components which can be in stoichiometric or non-stoichiometric amounts. The resulting complexes can be ionised, partially ionised, or non-ionised. For a review of such complexes, see J. Pharm. ScL, 64 (8), 1269-1288, by Haleblian (August 1975).

6. Components

Disclosed are the components to be used to prepare the disclosed compositions as well as the compositions themselves to be used within the methods disclosed herein. These and other materials are disclosed herein, and it is understood that when combinations, subsets, interactions, groups, etc., of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

7. Compounds and Compositions

Compounds and compositions have their standard meaning in the art. It is understood that wherever, a particular designation, such as a molecule, substance, cell, or reagent compositions comprising, consisting of, and consisting essentially of these designations are disclosed. Where appropriate wherever a particular designation is made, it is understood that the compound of that designation is also disclosed.

8. Chemical Terms

i. Aryl

The term “aryl” as used herein is a ring radical containing 6 to 18 carbons, or preferably 6 to 12 carbons, comprising at least one aromatic residue therein. Examples of such aryl radicals include phenyl, naphthyl, and ischroman radicals. Moreover, the term “aryl” as used throughout the specification and claims is intended to include both ^{“}unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. An aryl moiety with 1, 2, or 3 alkyl substituent groups can be referred to as “arylalkyl.”It will be understood by those skilled in the art that the moieties substituted on the “aryl” can themselves be substituted, as described above, if appropriate.

ii. Heteroatom

The term “heteroatom” as used herein refers to an atom of an element other than carbon or hydrogen.

iii. Heteroaryl

The term “heteroaryl” as used herein is an aryl ring radical as defined above, wherein at least one of the ring carbons, or preferably 1, 2, or 3 carbons of the aryl aromatic ring has been replaced with a heteroatom, which include but are not limited to nitrogen, oxygen, and sulfur atoms. Examples of heteroaryl residues include pyridyl, bipyridyl, furanyl, and thiofuranyl residues. Substituted “heteroaryl” residues can have one or more organic or inorganic substituent groups, or preferably 1, 2, or 3 such groups per ring, as referred to herein-above for aryl groups, bound to the carbon atoms of the heteroaromatic rings. The organic substituent groups can comprise from 1 to 12 carbon atoms, or from 1 to 6 carbon atoms, or from 1 to 4 carbon atoms.

iv. Heterocyclyl

The term “heterocyclyl” or “heterocyclic group” as used herein is a non-aromatic mono- or multi ring radical structure having 3 to 16 members, preferably 4 to 10 members, in which at least one ring structure include 1 to 4 heteroatoms (e.g. O, N, S, P, and the like). Heterocyclyl groups include, for example, pyrrolidine, benzodioxoles, oxolane, thiolane, imidazole, oxazole, piperidine, piperizine, morpholine, lactones, such as thiobutyrolactones, lactams, such as azetidiones, and pyrrolidiones, sultams, sultones, and the like. Moreover, the term “heterocyclyl” as used throughout the specification and claims is intended to include both unsubstituted heterocyclyls and substituted heterocyclyls; the latter denotes a ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. It will be understood by those skilled in the art that the moieties substituted on the “heterocyclyl” can themselves be substituted, as described above, if appropriate.

v. Carbocyclic

The term “carbocyclic” as used herein refers to a cyclic moiety in which all members forming the ring are carbon atoms.

vi. Alkyl

The term “alkyl” as used herein refers to a branched or unbranched saturated hydrocarbon moiety, which can optionally be cyclical or contain a cyclical portion. Alkyls comprise a saturated hydrocarbon moiety having from 1 to 24 carbons, 1 to 20 carbons, 1 to 15 carbons, 1 to 12 carbons, 1 to 8 carbons, 1 to 6 carbons, 1 to 4 carbon atoms, or 1 to 3 carbon atoms. It is understood that the term “alkyl” also encompasses linear, branched or cyclic hydrocarbon moieties having 1, 2, 3, 4, 5, 6, 7, 8, 9, 10, 11, 12, 13, 14, 15, 16, 17, 18, 19, 20, 21, 22, 23, or 24 carbon atoms. Examples of such alkyl radicals include methyl, ethyl, propyl, butyl, pentyl, hexyl, heptyl, octyl, n-propyl, iso-propyl, cyclopropyl, butyl, n-butyl, sec-butyl, t-butyl, cyclobutyl, amyl, t-amyl, n-pentyl, cyclopentyl, and the like. Lower alkyls comprise a noncyclic, saturated, straight or branched chain hydrocarbon residue having from 1 to 4 carbon atoms, i.e., C₁-C₄ alkyl.

Moreover, the term “alkyl” as used throughout the specification and claims is intended to include both “unsubstituted alkyls” and “substituted alkyls”; the latter denotes an alkyl radical analogous to the above definition, that is further substituted with one, two, or more additional organic or inorganic substituent groups. Suitable substituent groups include but are not limited to H, alkyl, alkenyl, alkynyl, hydroxyl, cycloalkyl, heterocyclyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. It will be understood by those skilled in the art that an “alkoxy” can be a substitutent of a carbonyl substituted “alkyl” forming an ester. When more than one substituent group is present then they can be the same or different. The organic substituent moieties can comprise from 1 to 12 carbon atoms, or from 1 to 6 carbon atoms, or from 1 to 4 carbon atoms. It will be understood by those skilled in the art that the moieties substituted on the “alkyl” chain can themselves be substituted, as described above, if appropriate.

vii. Alkenyl

The term “alkenyl” as used herein is an alkyl residue as defined above that also comprises at least one carbon-carbon double bond in the backbone of the hydrocarbon chain. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

viii. Alkynyl

The term “alkynyl” as used herein is an alkyl residue as defined above that comprises at least one carbon-carbon triple bond in the backbone of the hydrocarbon chain. Examples include but are not limited ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

ix. Cycloalkyl

The term “cycloalkyl” as used herein is a saturated hydrocarbon structure wherein the structure is closed to form at least one ring. Cycloalkyls typically comprise a cyclic radical containing 3 to 8 ring carbons, such as cyclopropyl, cyclobutyl, cyclopentyl, cyclopenyl, cyclohexyl, cycloheptyl and the like. Cycloalkyl radicals can be multicyclic and can contain a total of 3 to 18 carbons, or preferably 4 to 12 carbons, or 5 to 8 carbons. Examples of multicyclic cycloalkyls include decahydronapthyl, adamantyl, and like radicals.

Moreover, the term “cycloalkyl” as used throughout the specification and claims is intended to include both “unsubstituted cycloalkyls” and “substituted cycloalkyls”, the later denotes an cycloalkyl radical analogous to the above definition that is further substituted with one, two, or more additional organic or inorganic substituent groups that can include but are not limited to hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. When the cycloalkyl is substituted with more than one substituent group, they can be the same or different. The organic substituent groups can comprise from 1 to 12 carbon atoms, or from 1 to 6 carbon atoms, or from 1 to 4 carbon atoms.

x. Cycloalkenyl

The term “cycloalkenyl” as used herein is a cycloalkyl radical as defined above that further comprises at least one carbon-carbon double bond. Examples include but are not limited to cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl and the like.

xi. Lower Hydrocarbon Moiety

The term “hydrocarbon moiety” as used herein refers to hydrocarbons, saturated or unsaturated, linear or branched or cyclic, substituted or unsubstituted, having up to eight carbons.

xii. Alkoxy

The term “alkoxy” as used herein refers to an alkyl residue, as defined above, bonded directly to an oxygen atom, which is then bonded to another moiety. Examples include methoxy, ethoxy, n-propoxy, iso-propoxy, n-butoxy, t-butoxy, iso-butoxy and the like. The term “lower alkoxy” as used herein refers to an alkoxy residue having up to eight carbons in the alkyl radical.

xiii. Amino

The term “amino” as used herein is a moiety comprising a N radical substituted with zero, one or two organic substituent groups, which include but are not limited to alkyls, substituted alkyls, cycloalkyls, aryls, or arylalkyls. If there are two substituent groups they can be different or the same. Examples of amino groups include, —NH₂, methylamino (—NH—CH₃); ethylamino (—NHCH₂CH₃), hydroxyethylamino (—NH—CH₂CH₂OH), dimethylamino, methylethylamino, diethylamino, and the like.

xiv. Mono-Substituted Amino

The term “mono-substituted amino” as used herein is a moiety comprising an NH radical substituted with one organic substituent group, which include but are not limited to alkyls, substituted alkyls, cycloalkyls, aryls, or arylalkyls. Examples of mono-substituted amino groups include methylamino (—NH—CH₃); ethylamino (—NHCH₂CH₃), hydroxyethylamino (—NH—CH₂CH₂OH), and the like.

xv. Di-Substituted Amino

The term “di-substituted amino” as used herein is a moiety comprising a nitrogen atom substituted with two organic radicals that can be the same or different, which can be selected from but are not limited to aryl, substituted aryl, alkyl, substituted alkyl or arylalkyl, wherein the terms have the same definitions found throughout. Some examples include dimethylamino, methylethylamino, diethylamino and the like.

xvi. Acyl

The term “acyl” as used herein is a R—C(O)— residue having an R group containing 1 to 8 carbons. The term “acyl” encompass acyl halide, R—(O)-halogen. Examples include but are not limited to formyl, acetyl, propionyl, butanoyl, iso-butanoyl, pentanoyl, hexanoyl, heptanoyl, benzoyl and the like, and natural or un-natural amino acids.

xvii. Acyloxy

The term “acyloxy” as used herein is an acyl radical as defined above directly attached to an oxygen to form an R—C(O)O— residue. Examples include but are not limited to acetyloxy, propionyloxy, butanoyloxy, iso-butanoyloxy, benzoyloxy and the like.

xviii. Azide

As used herein, the term “azide”, “azido” and their variants refer to any moiety or compound comprising the monovalent group —N₃ or the monovalent ion —N₃.

xix. Benzo Group

The terms “benzo”, “benzo group,” and “fused benzo group” as used herein refers to a phenyl group that has in common with another moiety two neighboring carbon atoms that are bonded to one another. In particular, these and like terms as used herein refer to the sharing of two neighboring phenyl ring carbons with another cyclic moiety.

xx. Bond

The term “bond” as used herein has its usual and ordinary meaning in organic chemistry.

xxi. Together Form a Bond

The term “together form a bond” as used herein with respect to two labeled indices in a figure means that the indices are in fact absent and that the neighbors shown as connected to either side of those paired indices are in fact bonded to each other. E.g., where the structure shows a phenyl ring connected as [Ph figure]-a-b-c, and it is said herein that “a and b together form a bond,” this indicates that a and b are absent, and that c has a covalent bond to the phenyl ring at the ring carbon to which a is shown as being attached.

xxii. Bridge

The term “bridge” as used herein refers to a cyclic moiety in which two atoms that are part of a covalent sequence of atoms are each bonded to the same substituent such that it defines a bridge between them, and such that together with the covalent sequence of atoms defines a cyclic moiety.

xxiii. Together Form a Bridge

The term “together form a bridge” as used herein with respect to respective substituents on two atoms refers to the same phenomenon as defined herein for the term “bridge”.

xxiv. Electron Withdrawing Group

The term “electron withdrawing” as used herein has its usual and ordinary meaning in organic chemistry, and refers to highly electronegative substituents such as: halides such as fluoride, chloride, and the like; pseudohalides such as cyanide, cyanate, thiocyanate, and the like; nitro and nitroso groups and the like; sulfate groups, tosyl groups and the like; doubly bonded oxygen; and other highly electronegative substituents.

xxv. Haloalkyl

The term “haloalkyl” as used herein an alkyl residue as defined above, substituted with one or more halogens, preferably fluorine, such as a trifluoromethyl, pentafluoroethyl and the like.

xxvi. Haloalkoxy

The term “haloalkoxy” as used herein refers to a haloalkyl residue as defined above that is directly attached to an oxygen to form trifluoromethoxy, pentafluoroethoxy and the like.

xxvii. Halogen or Halo or Halide

The term “halo” or “halogen” or “halide” as used herein refers to a fluoro, chloro, bromo, or iodo group.

xxviii. In any Order

The term “in any order” as used herein refers to a linear series having a plurality of members, wherein the members can be arranged in any order relative to one another in the series.

xxix. Respective

The term “respective” as used herein with respect to substituents and the atoms on which they are substituted and designated by a common index refers to the independent identity of such substituents relative to one another, and indicates that each particular atom is treated site is treated independently. For example, for a series of methylene atoms in which each is substituted by R^{b}, the term “substituted by a respective R^{b}” indicates that the identity of R^{b} is independent and potentially unique for each substituted methylene. In such contexts herein the term “respective” is used for the sake of verbal economy in designating the widest scope of permutation in sequences.

xxx. Linker

The term “linker” as used herein refers to a covalently bonded sequence of from one to eight atoms, in which one end of the sequence is covalently bonded to a first moiety and the other end of the sequence is covalently bonded to a second moiety; the structures of the first and second moieties can be like or unlike one another.

xxxi. Moiety

The term “moiety” as used herein refers to part of a molecule (or compound, or analog, etc.). A “functional group” is a specific group of atoms in a molecule. A moiety can be a functional group or can include one or more functional groups.

xxxii. Ester

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

xxxiii. Carbonate Group

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

xxxiv. Keto Group

The term “keto group” as used herein is represented by the formula —C(O)R, where R is an alkyl, alkenyl, alkynyl, aryl, aralkyl, cycloalkyl, halogenated alkyl, or heterocycloalkyl group described above.

xxxv. Aldehyde

The term “aldehyde” as used herein is represented by the formula —C(O)H or —R—C(O)H, wherein R can be as defined above alkyl, alkenyl, alkoxy, aryl, heteroaryl, cycloalkyl, cycloalkenyl, heterocycloalkyl, or heterocycloalkenyl group described above.

xxxvi. Carboxylic Acid

The term “carboxylic acid” as used herein is represented by the formula —C(O)OH.

xxxvii. Carbonyl Group

The term “carbonyl group” as used herein is represented by the formula C═O.

xxxviii. Ether

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

xxxix. Urethane

The term “urethane” as used herein is represented by the formula —OC(O)NRR′, where R and R′ can be, independently, hydrogen, an alkyl, alkenyl, alkynyl, aryl, aralkyl, cycloalkyl, halogenated alkyl, or heterocycloalkyl group described above.

xl. Methylene

The term “methylene” as used herein refers to a carbon atom in series —C(R)(R′)— wherein R and R′ can be, independently, hydrogen, a lower hydrocarbon moiety, an electron withdrawing group, aryl, aralkyl, alkaryl, halogenated alkyl, alkoxy, heteroaryl or heterocycloalkyl group described above. In particular embodiments R and R′ are selected from hydrogen and unsubstituted lower hydrocarbon moieties.

xli. Silyl Group

The term “silyl group” as used herein is represented by the formula —SiRR′R″, where R, R′, and R″ can be, independently, hydrogen, an alkyl, alkenyl, alkynyl, aryl, aralkyl, cycloalkyl, halogenated alkyl, alkoxy, or heterocycloalkyl group described above.

xlii. Sulfo-Oxo Group

The term “sulfo-oxo group” as used herein is represented by the formulas —S(O)₂R, —OS(O)₂R, or, —OS(O)₂OR, where R can be hydrogen or as defined above an alkyl, alkenyl, alkynyl, aryl, aralkyl, cycloalkyl, halogenated alkyl, or heterocycloalkyl group described above.

9. Inhibit

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibiting PPAR” means hindering or restraining the amount of PPAR activity that takes place relative to a standard or a control.

10. Or

The word “or” or like terms as used herein means any one member of a particular list and also includes any combination of members of that list.

11. PPAR-Mediated Disease or Condition

The term “PPAR-mediated disease or condition” refers to any disease or condition in which PPAR or PPAR activity plays a role.

12. PPARγ-Mediated Disease or Condition

The term “PPARγ-mediated disease or condition” refers to any disease or condition in which PPARγ or PPARγ activity plays a role.

13. Pro-Drug

The term “pro-drug or prodrug” is intended to encompass compounds which, under physiologic conditions, are converted into therapeutically active agents. A common method for making a prodrug is to include selected moieties which are hydrolyzed under physiologic conditions to reveal the desired molecule. In other embodiments, the prodrug is converted by an enzymatic activity of the host animal.

14. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

15. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

16. Salt(s) and Pharmaceutically Acceptable Salt(s)

The disclosed compounds can be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also can be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I or II with an acid whose anion, or a base whose cation, is generally considered suitable for human consumption. Pharmaceutically acceptable salts are particularly useful as products of the disclosed methods because of their greater aqueous solubility relative to the parent compound. For use in medicine, the salts of the disclosed compounds are non-toxic “pharmaceutically acceptable salts.” Salts encompassed within the term “pharmaceutically acceptable salts” refer to non-toxic salts of the disclosed compounds which are generally prepared by reacting the free base with a suitable organic or inorganic acid.

Suitable pharmaceutically acceptable acid addition salts of the disclosed compounds when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate. Furthermore, where the disclosed compounds carry an acidic moiety, suitable pharmaceutically acceptable salts thereof can include alkali metal salts, i.e., sodium or potassium salts; alkaline earth metal salts, e.g., calcium or magnesium salts; and salts formed with suitable organic ligands, e.g., quaternary ammonium salts. In other embodiments, base salts are formed from bases which form non-toxic salts, including aluminum, arginine, benzathine, choline, diethylamine, diolamine, glycine, lysine, meglumine, olamine, tromethamine and zinc salts.

Organic salts can be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl (CrC₆) halides (e.g., methyl, ethyl, propyl, and butyl chlorides, bromides, and iodides), dialkyl sulfates (i.e., dimethyl, diethyl, dibuytl, and diamyl sulfates), long chain halides (i.e., decyl, lauryl, myristyl, and stearyl chlorides, bromides, and iodides), arylalkyl halides (i.e., benzyl and phenethyl bromides), and others.

In some embodiments, hemisalts of acids and bases can also be formed, for example, hemisulphate and hemicalcium salts.

The disclosed compounds and their salts can exist in both unsolvated and solvated forms.

17. Solvate

The compounds herein, and the pharmaceutically acceptable salts thereof, can exist in a continuum of solid states ranging from fully amorphous to fully crystalline. They can also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent can be isotopically substituted (e.g., D₂O, d₆-acetone, d₆-DMSO).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm.

The compounds herein, and the pharmaceutically acceptable salts thereof, can also exist as multi-component complexes (other than salts and solvates) in which the compound and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals can be prepared by melt crystallization, by recrystallization from solvents, or by physically grinding the components together. See, e.g., O. Almarsson and M. J. Zaworotko, Chem. Commun., 17:1889-1896 (2004). For a general review of multi-component complexes, see J. K. Haleblian, J. Pharm. Sci. 64(8):1269-88 (1975).

18. Subject

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

19. Tautomer

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

20. Therapeutically Effective

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

21. Treat, Treating, Treatment or Therapy

In the context of a subject “Treating” or “treatment” or “therapy” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. The term treat can also mean to prevent a disease or symptom from occurring in a subject at risk of developing a disease.

### EXAMPLES

### I. Example 1

1. Introduction

Nuclear receptors represent an important class of receptor targets for drug discovery. The peroxisome proliferator-activated receptors (PPARs) are ligand activated transcription factors that belong to the nuclear receptor superfamily and play very important roles in multiple physiological pathways. A new class of small molecules were designed and synthesized based on a fluorescent compound YL-1-04-02 targeting PPARs. The PPAR isotype screening demonstrates that these compounds can serve as a new class of antagonists of PPARs. Representative compound YL-1-38-1 exhibits PPARγ-preferential antagonistic activity.

2. Results

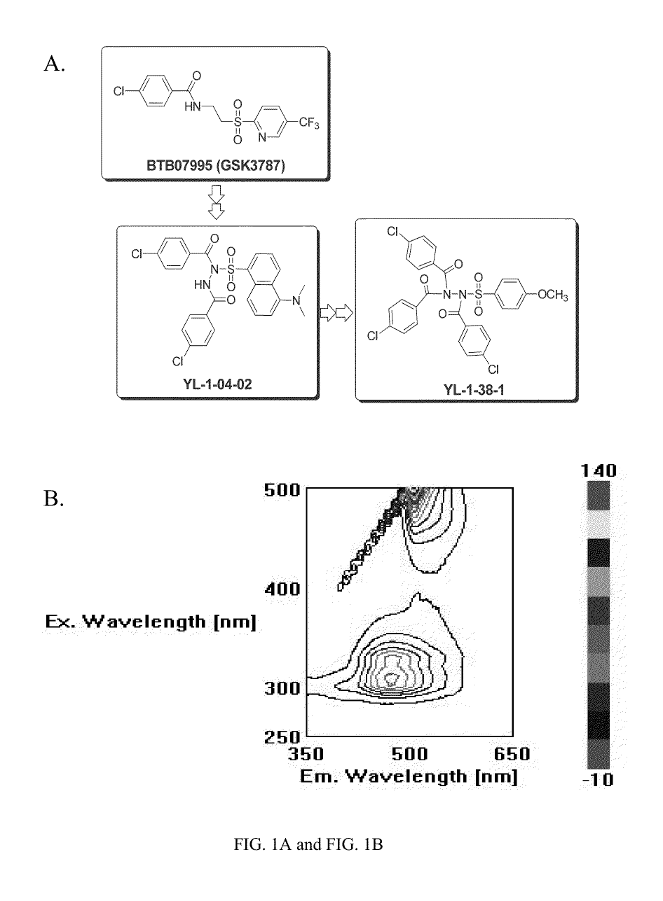

GSK3787(BTB07995) (Shearer, G. B., et al. J Med Chem 53:1857-1861, 2010) was identified as a potent and selective ligand for PPARδ with good pharmacokinetic properties. However, this compound functioned as a suicide inhibitor by covalent bonding to Cys249 in the ligand-binding pocket of PPARδ through its trifluoromethylpyridyl group. Due to this key limitation, to make a reversible, fluorescent inhibitor of PPARs, the structure of BTB07995 was modified. This resulted in the discovery of YL-1-04-02. Based on the biological data of YL-1-04-02, a series of derivatives were synthesized targeting PPARγ (FIG. 1A). FIG. 1B shows the dansyl moiety present in compound YL-1-04-2 allows it to visibly fluoresce at 480 nm when excited at 306 nm.

i. Biological Evaluation:

a. Functional Assay:

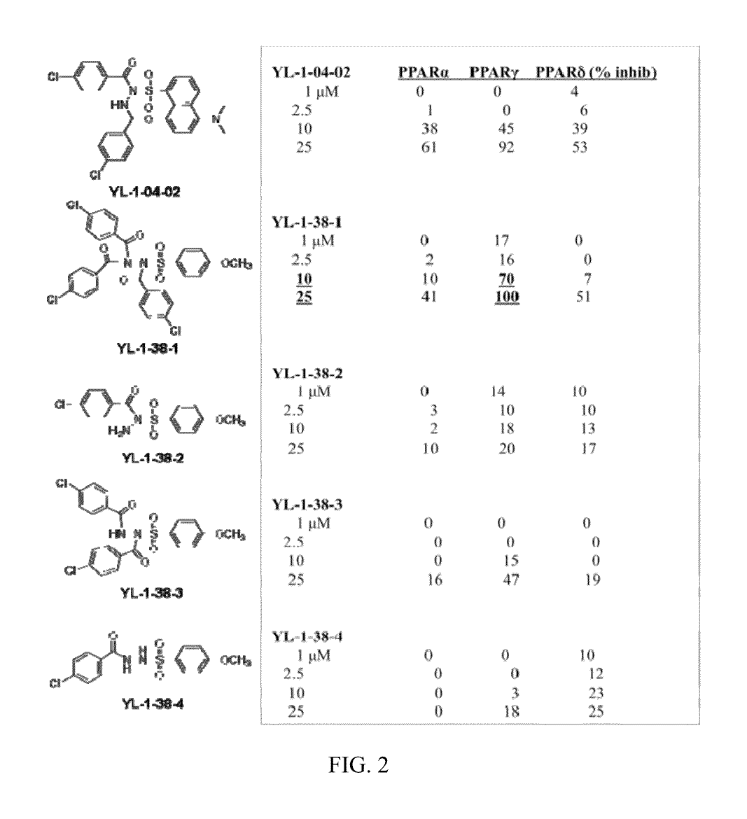

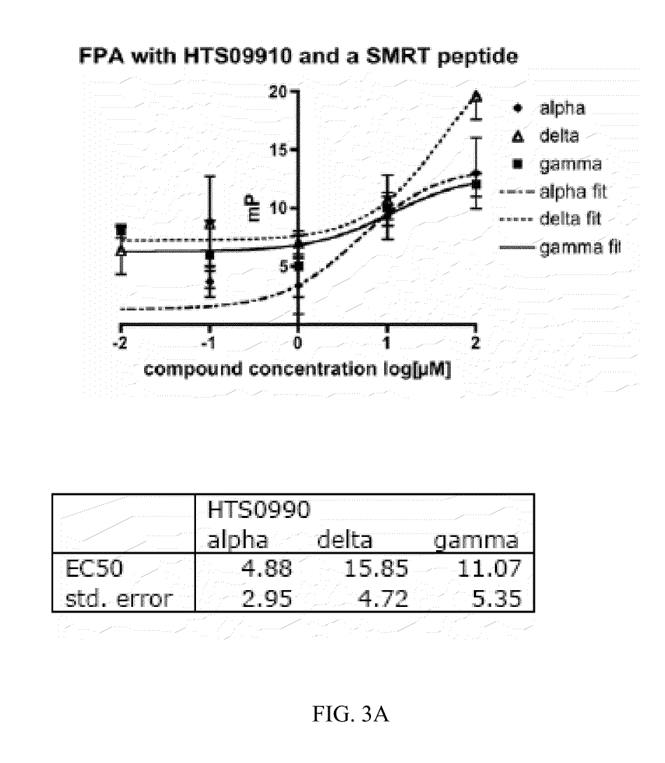

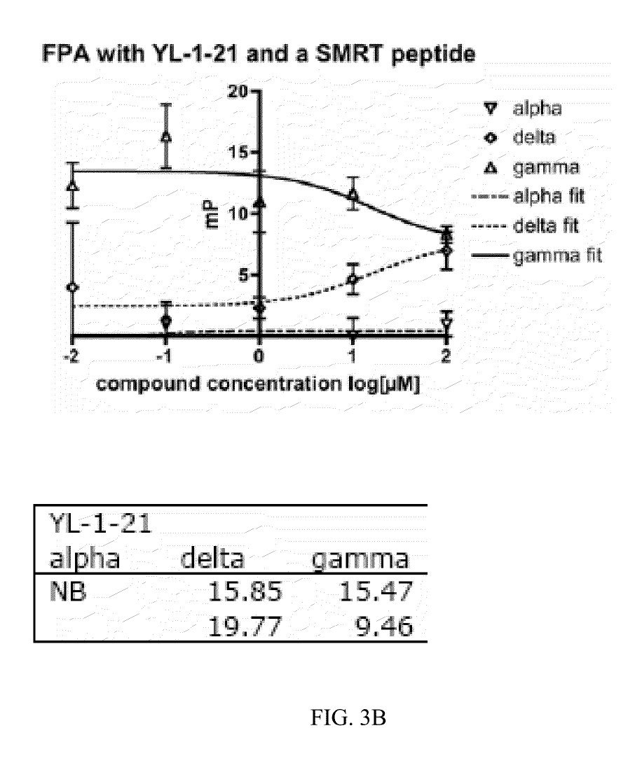

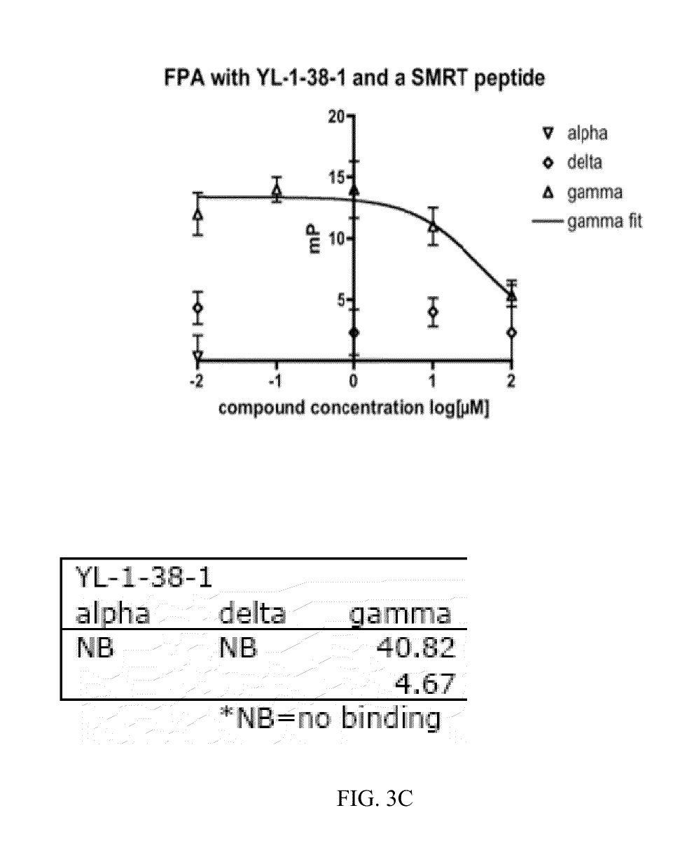

Compounds were tested for their ability to inhibit activation of each PPAR in the presence of 1 μM agonist (WY14643, PPARα; GW7845, PPARγ; GW501516, PPARδ). FIG. 2 showed the percent inhibition of PPAR stimulation by the respective agonists. YL-1-38-1 indicated promising PPARγ selectivity (FIG. 2), which was then confirmed by FP experiments (FIG. 3).

293T cells were grown in 24-well plates in DMEM containing 10% fetal calf serum; after 24 hr, medium was replaced with DMEM containing 10% delipidated fetal calf serum (Sigma-Aldrich Chemical Co.). Cells were transfected using calcium phosphate precipitation (Promega) with the appropriate combination of luciferase reporter plasmid (p3XPPRE-TK-Luc for PPARγ or pG5Luc for Gal4 fusion proteins), vector expressing the gene of interest and empty control vector. After 24 hr, cells were treated with 1.0 μM agonist (WY14643, PPARα; GW7485, PPARγ; GW501516, PPARδ).

b. Binding Assay

Fluorescent Polarization (FP) assays were established using the fluorescent corepressor peptides, NCoR1 (residues 2251-2275, FITC-GHSFADPASNLGLEDIIRKALMGSF, SEQ ID NO:2, Genbank accession NP_{—}006302) and SMRT (residues 1316-1337, FITC-TNMGLEAIIRKALMGKYDQWEE, SEQ ID NO:3, Genbank accession AAC50236), and recombinant PPARδ and γ ligand-binding domains (LBDs) and full-length PPARδ (Cayman Chemicals)). For PPARγ screening, a fluorescent ligand supplied by Cayman Chemicals was also used. All compounds were dissolved in DMSO as 10 mM stock solutions and the final DMSO content in the assay was <1%. A TECAN Ultra 485 multi-functional microplate reader and GraphPad prism 4 software were used for measurements and analysis, respectively. GW501516 and eicosapentaenoic Acid (EPA) were used as controls, and their binding constants were within the expected values. Although GW501516 is a selective PPARδ agonist, it has affinity for PPARα and PPARγ at 1000-fold higher concentrations (˜1 μM) (Shearer B. G., et al. Curr Med Chem 10:267-80, 2003).

PPAR antagonists are expected to enhance the affinities of the corepressor peptides, and therefore, FP should increase as the compound concentration increases. Agonists would be expected to weaken the affinity of the same co-repressor peptide. Although this effect occurs for PPARγ, the dissociation of corepressor peptides varies for PPARα and PPARδ due to altered presentations of the overlapping coactivator/corepressor binding surfaces (Stanley T. B. et al. Biochemistry 42:9278-87, 2003). Compounds were screened initially against PPARγ and PPARδ at 1 and 100 μM. If binding was observed, titration experiments from 10 nM to 100 μM were carried out in triplicate with all three PPARs. One hundred compounds were tested and 12 compounds were identified with binding activity. Examples of FP assays for compounds HTS09910, YL-1-21 and YL-1-38-1 are shown in FIG. 3, where YL-1-38-1 shows selective binding to PPARγ. HTS09910 enhanced FP to all three PPARs (FIG. 3A), while YL-1-21 and YL-1-38-1 weakened the affinity of the peptide to PPARγ (FIG. 3B and FIG. 3C, respectively). For screening, either enhancement or weakening of FP was considered active.

FP (Fluorescent Polarization) assay for compound YL-1-38-1 is shown in FIG. 3. YL-1-38-1 shows selective binding to PPARγ, it weakened the affinity of the peptide to PPARγ in a dose dependent manner (FIG. 3). For screening, either enhancement or weakening of FP was considered active. The EC50 value of YL-1-38-1 is determined.

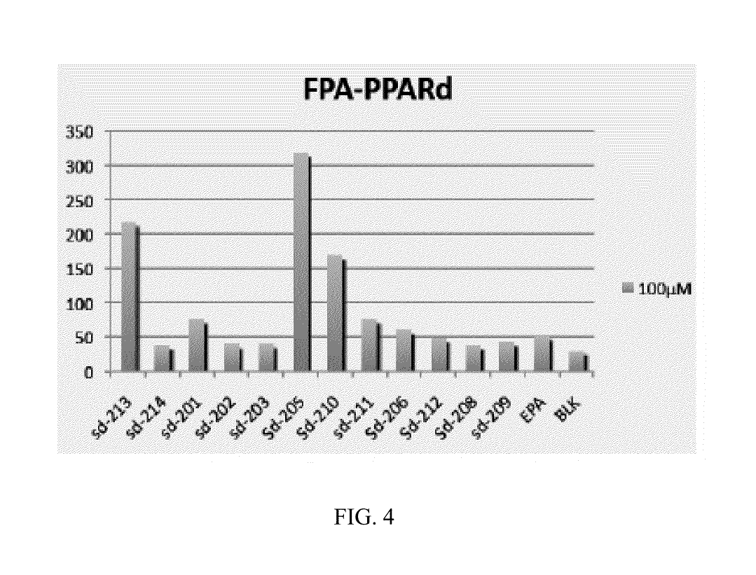

Fifteen thousand (15,000) additional compounds were screened in silico against PPARδ in its expected antagonist conformation and 150 compounds were selected for FPA screening. Of the 150 compounds, 51 have been received and 34 have been evaluated. Three compounds have demonstrated FP activity so far (FIG. 4) but none were sufficiently selective against PPARδ in reporter assays. Fifteen additional compounds are in the process of being evaluated and we are awaiting 74 compounds.

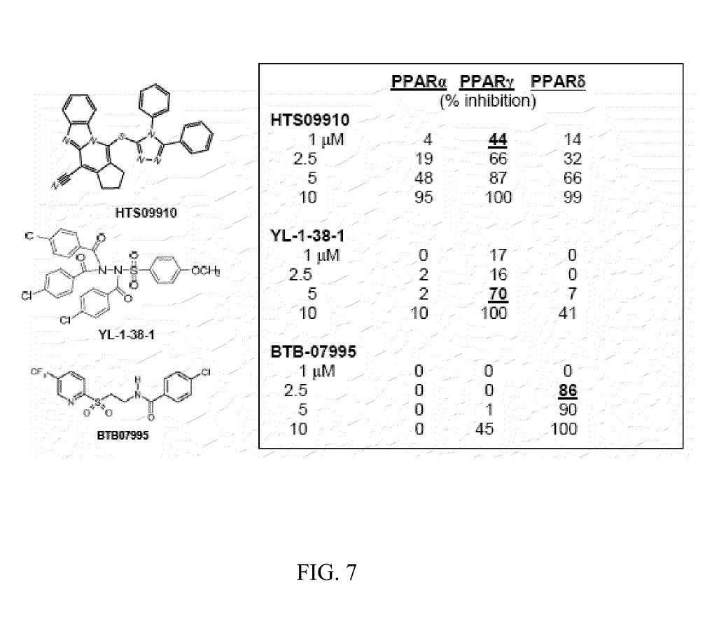

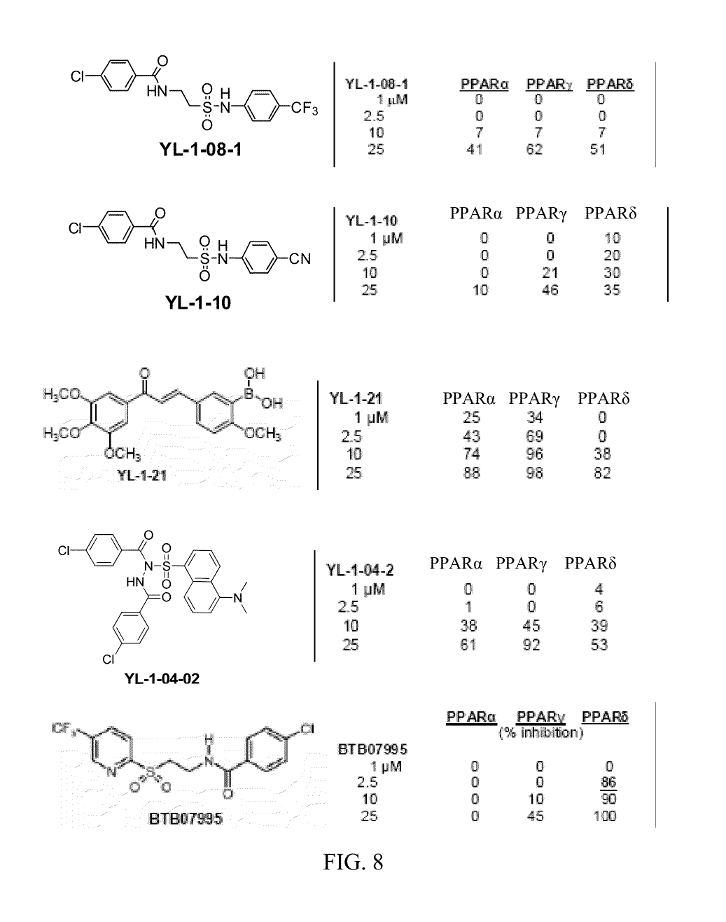

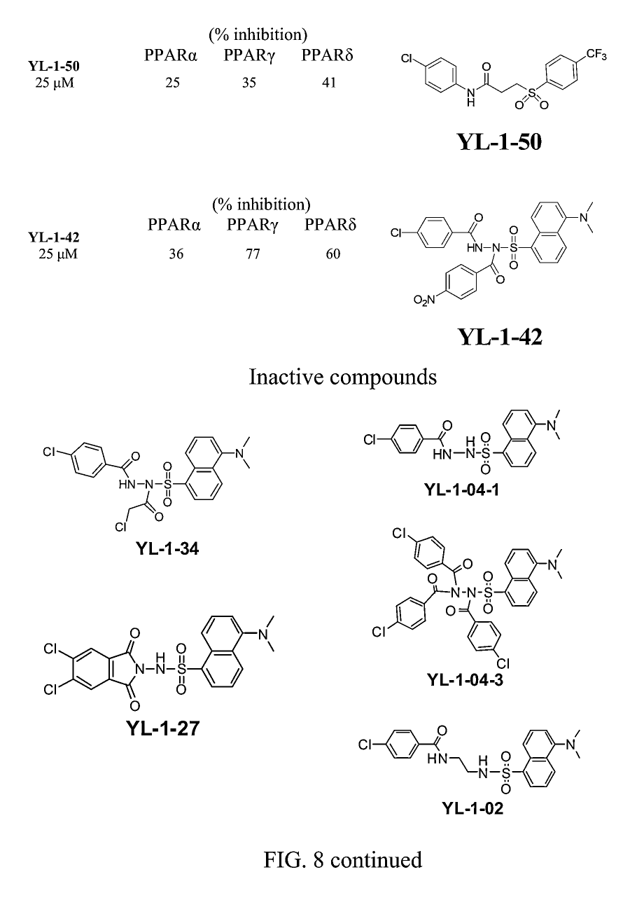

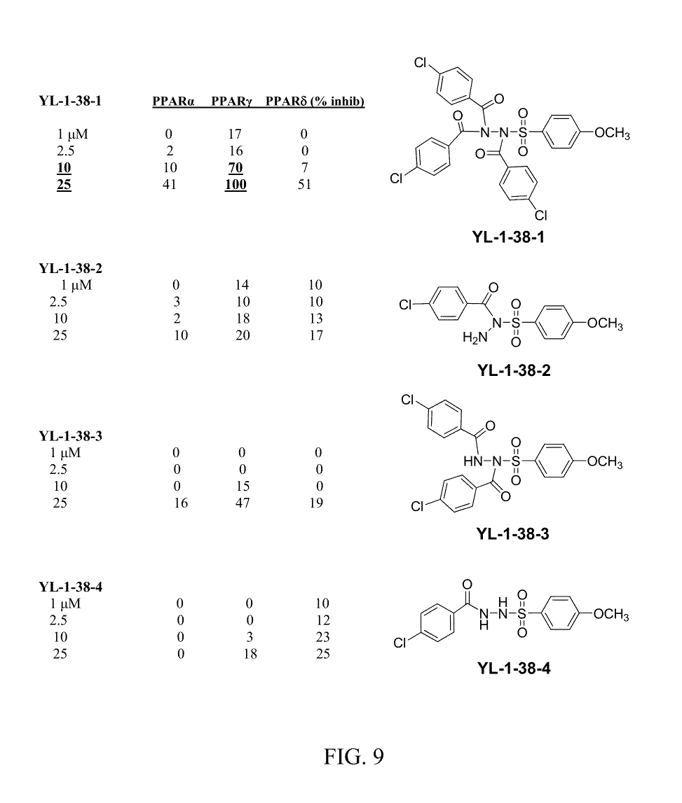

One hundred thirty eight (138) compounds were screened by reporter assays for PPARα, PPARγ and PPARδ activity, and two PPARγ antagonists and one PPARδ antagonist were identified (FIG. 7). Assays of 30 compounds structurally related to YL-1-38-1 and BTB07995, some of which are shown in FIG. 8 and FIG. 9, indicated that only YL-1-38-1 and BTB07995 possessed PPARγ and PPARδ selectively, respectively.

c. Docking

Virtual screening was performed against 56,000 compounds from the Maybridge library that targeted the ligand binding domain (LBD) of PPARγ, and 10 conformations of each compound were docked to the LBD using Autdock4 software (Scripps Institute). Sixty (60) of the top ranked compounds were ordered from Maybridge, and 58 were available for evaluation.

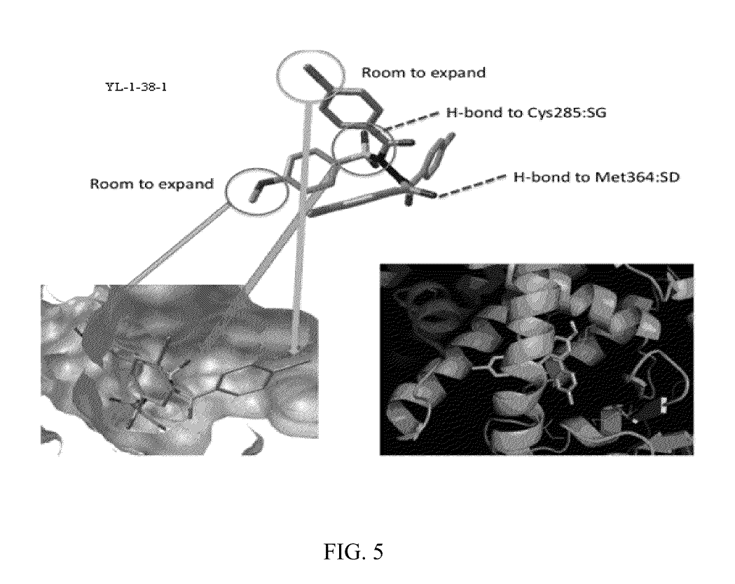

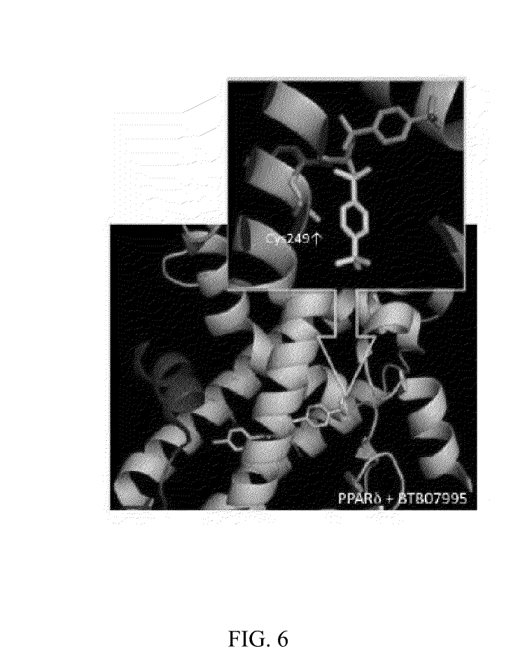

The binding of YL-1-38-1 with PPARγ ligand binding domain (autoDock software) shows that it utilized all three binding arms of the PPARγ LBD. The further modification of each substituent should increase their interaction with the LBD to enhance their affinity and selectivity (FIG. 5). The trifluoromethyl-pyridyl group of the PPARδ antagonist, BTB07995, is expected to be conformationally flexible within either of the two arms of the PPARδ LBD (FIG. 6). Docked structures can be used as a guide to establish SAR for candidate compounds.

ii. Analogs

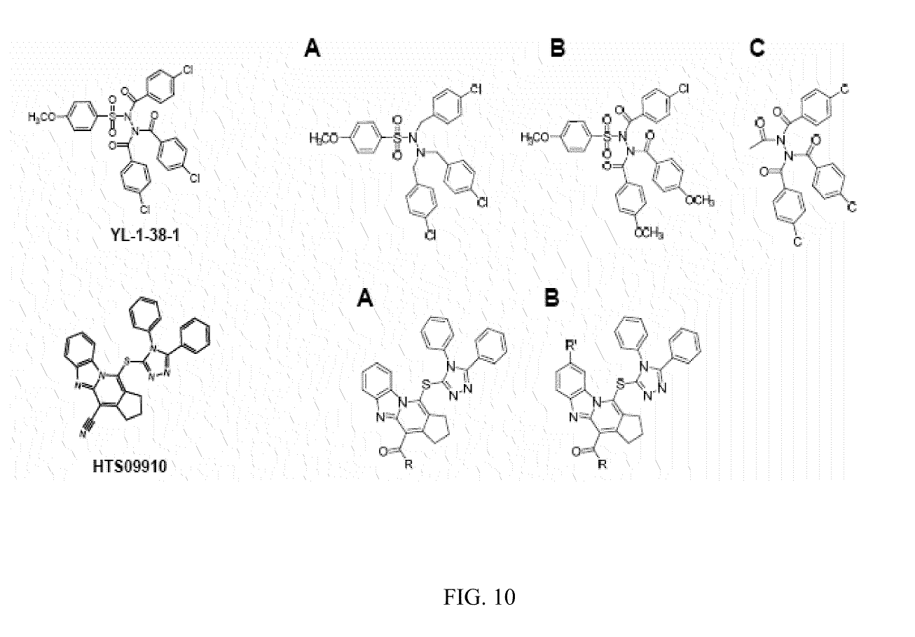

Three pharmacophores, HTS09910, YL-1-38-1 and BTB07995 have been identified and can be further modified to increase potency against their respective PPAR for in vitro evaluation and eventually in vivo testing. Analogs of YL-1-38-1 and HTS09910 are shown in FIG. 10.

3. Conclusion

A fluorescent compound, YL-1-04-02, and its derivative YL-1-38-1 were identified as new antagonists of PPARγ. The data demonstrates that these compounds can serve as a new class of antagonists of PPARγ.

### J. Example 2

### Identifying PPARγ and PPARδ Antagonists

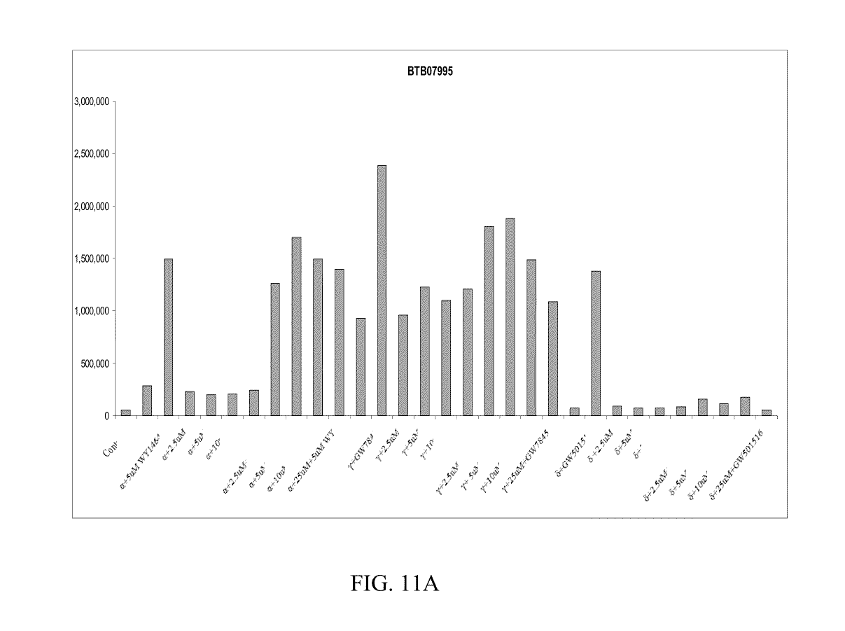

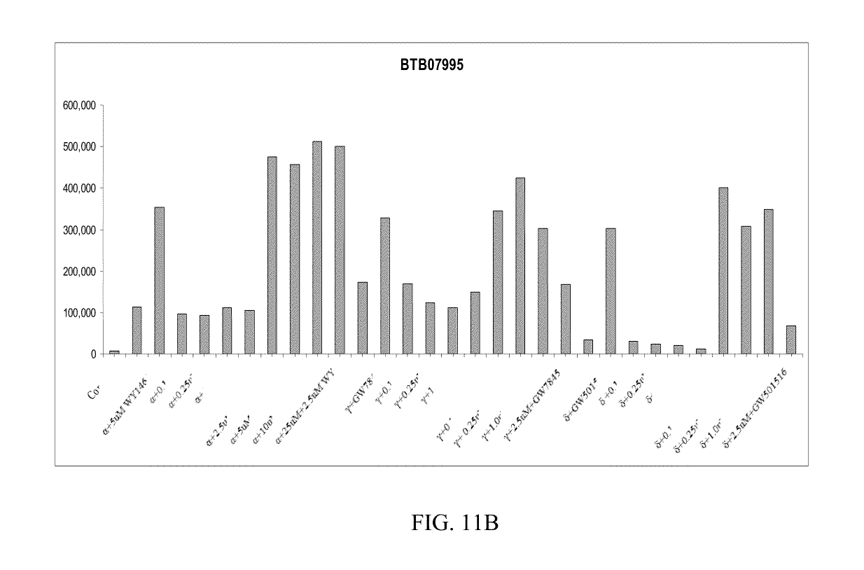

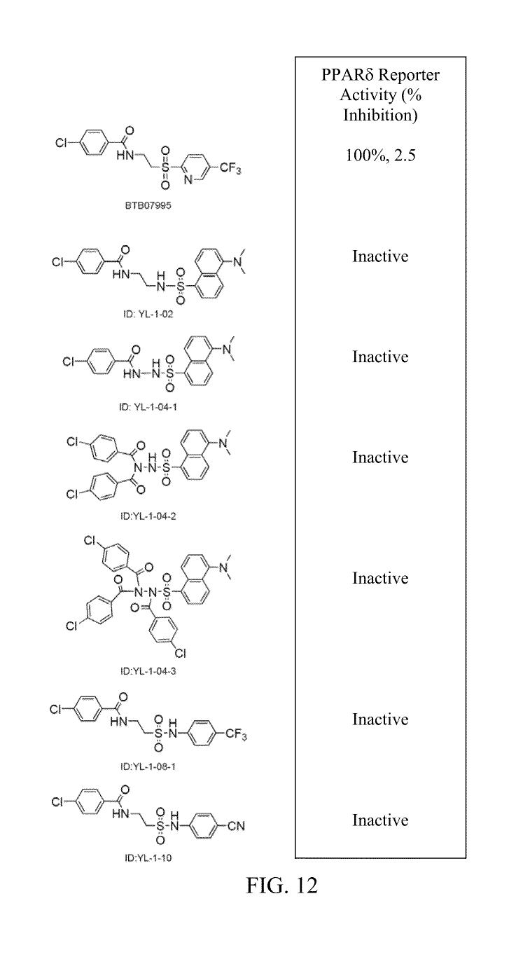

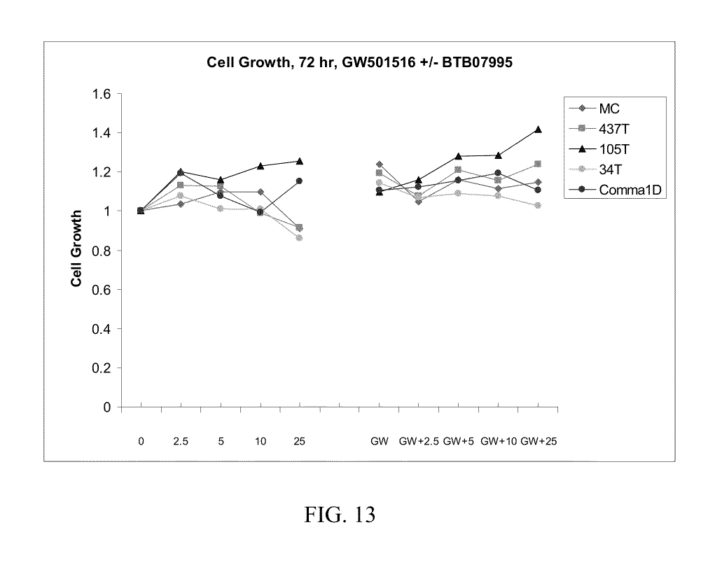

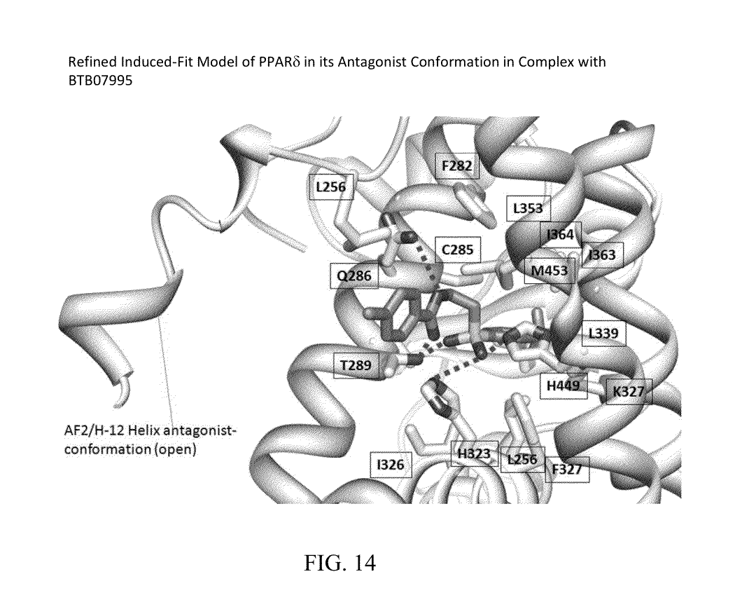

Two structure-based drug design approaches were taken. Based on a Maybridge chemical library, BTB07995 was identified as a PPARδ antagonist by reporter gene assay. FIG. 11 shows that BTB07995 is a selective antagonist of PPARδ, and is not an agonist for PPARα, PPARδ and PPARγ. BTB07995 was not cytotoxic to four mouse mammary tumor cell lines and one mouse mammary epithelial cell (FIG. 12).

It was further determined that replacement of the trifluoromethylpyridinyl group in BTB07995 with a dansyl group, as well as the position of the sulfoxide adjacent to the trifluoromethylpyridinyl group were critical for PPARδ antagonism.

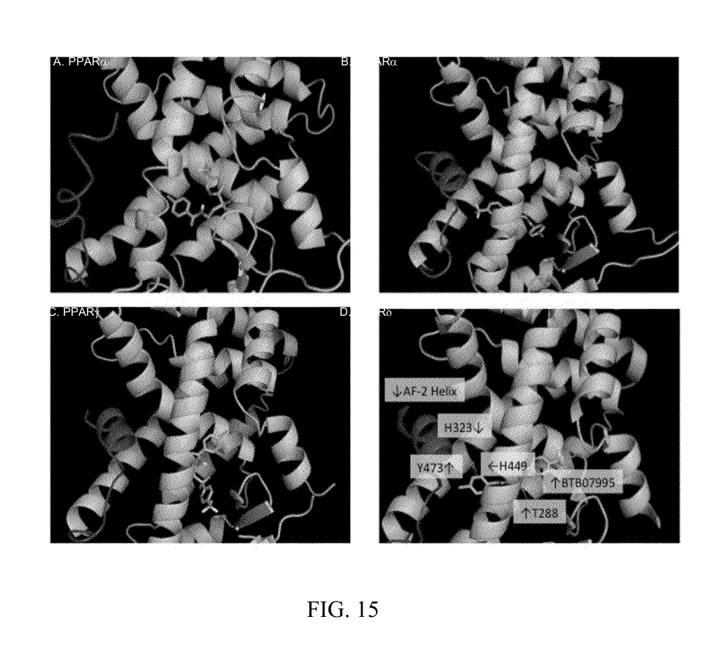

Shown in FIG. 15 are four PPAR complex structures: PPARα in an agonist and an antagonist bound form (FIGS. 15A, B), PPARγ in an agonist-bound form (FIG. 15C) and PPARδ in an agonist-bound form (FIG. 15D) that were selected from the RCSB Protein Data Bank; receptor molecules were extracted removing all ligands. BTB07995 was docked with 10 conformations of each receptor using AutoDock 4.1 (The Scripps Research Institute, La Jolla, Calif.). Since BTB07995 is a flexible linear molecule, it was found to dock to PPARs in a variety of conformations with relatively small binding energy differences among them. One of the most stable complexes was found between PPARδ and BTB07995, and BTB07995 was stretched across the common ligand binding site (FIG. 15D). This virtual binding result is in agreement with a biological assay, which showed that BTB07995 selectively inhibits PPARδ, but not to PPARα or PPARγ.

To test if BTB07995 is engaged with PPARδ in the known antagonistic interaction (as seen in PPARα with its antagonist GW6471), BTB07995 was docked to PPARδ after removal of the AF-2 helix. Surprisingly, the interaction of BTB07995 to PPARδ without the AF-2 helix was weaker than that to PPARδ in its agonist conformation (as seen in PPARδ with its agonist GW2331). This contradictory result indicates that more subtle interactions and conformational changes dictate the switch between agonistic and antagonistic conformations and computational model alone may not be able to distinguish them well.

### K. Example 3

### Screening and Analysis of PPARγ and PPARδ Antagonists

Disclosed herein are PPARγ and PPARδ antagonists. Virtual screening for PPARγ was conducted against 56,000 compounds from the Maybridge library that targeted the ligand binding domain (LBD) of PPARγ, and 10 conformations of each compound were docked to the LBD using Autdock4 software (Scripps Institute). Sixty of the top ranked compounds were ordered from Maybridge, and 58 were available for evaluation. Fluorescent Polarization (FP) assays were established with the tagged co-repressor peptides NCoR1 and SMRT, the recombinant PPARα and PPARγ ligand-binding domains and full-length PPARδ. Table 1 presents binding and reporter data for all new analogs tested, where Sd-107-10 has exhibited the greatest selectivity for PPARγ, although not highly potent. Sd-107-10 interacts in the PPARγ LBD adjacent to helix 12, locking it into the antagonist co-repressor conformation (FIG. 16). Sd-107 and its analogs are disclosed herein. The chemical structure is shown below.

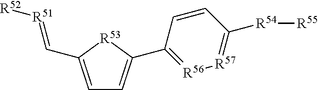

In some forms R⁵¹ can be a heterocyclic structure having two substituents selected from ═O and ═S. In some forms R⁵¹ can be a 5 membered heterocyclic structure having two substituents selected from ═O and ═S. In some forms R⁵¹ can be pyrazolidine-3,5,dione, 2-thioxothiazolidin-4-1, 2-thioxooxazolidin-4-1, thiazolidine-2,4-dione or 5-thioxopyrazolidin-3-1.

In some forms R⁵² can be substituted or unsubstituted aryl, substituted or unsubstituted heteroaryl, substituted or unsubstituted cycloalkyl, substituted or unsubstituted heterocyclyl, 1-methylcyclopropanecarboxylate C₁-C₆ alkyl,

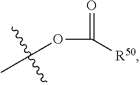

C₁-C₃ alkoxy, halo, C₁-C₃ haloalkyl, cyano or nitro. In some forms R⁵² can be phenyl, ethyl, butyl, cyclohexyl, biphenyl, phenoxybenzyl propyl 1-methylcyclopropanecarboxylate or halogenated benzene. In some forms R⁵² can be fluoro substituted benzene.

In some forms R⁵³ can be O, S or NH. In some forms R⁵³ can be O.

In some forms R⁵⁶ can be CH and R⁵⁷ can be CH. In some forms R⁵⁶ can be N and R⁵⁷ can be CH. In some forms R⁵⁶ can be CH and R⁵⁷ can be N.

In some forms R⁵⁴ can be —SO₂—, —NH—, —S(O)₂NH—, —NHCH₂—, —NHCH₂CH₂—, —NHCH₂CH₂CH₂—, —NHCOO—, —SO₂NHCOO— or —SO₂NHC(O)—. In some forms R⁵⁴ can be —SO₂— or —S(O)₂NH—.

In some forms R⁵⁵ can be H, C₁-C₃ alkyl, heteroaryl, heterocyclyl, aryl or cycloalkyl. In some forms R⁵⁵ can be H, C₁-C₃ alkyl, phenyl, pyrrole imidazole, oxazole, thiazole or triazole.

YL-1-38-1 was initially identified as a PPARγ antagonist, but additional dose-response assays indicate it is a pan inhibitor (Table 1). Eight analogs of YL-1-38-1 were synthesized, YL-1-68-1, YL-1-68-2, YL-1-69, YL-1-80, YL-1-81, YL-1-83, YL-1-87 and YL-1-88, which have been screened for PPAR binding (FIG. 17) and reporter activity (Table 1). Of these compounds, YL-1-83 is a weak PPARγ antagonist. Docking of YL-1-83 to the target binding site near the AF-2 helix of PPARγ is shown in FIG. 18.

An additional 15,000 compounds were screened in silico against PPARδ in its expected antagonist conformation. 150 compounds were selected for FP screening, and of these 51 were available. Three compounds demonstrated FP activity, but none were sufficiently selective against PPARδ in reporter assays. One PPARδ antagonist has been identified from the Maybridge library, BTB07995, and it is being evaluated in a PPARδ-dependent gastric cancer mouse model by MRI imaging to see if it blocks tumor initiation (Pollock C B, et al. Induction of metastatic gastric cancer by peroxisome proliferator-activated receptor-delta activation. PPAR Res. 2010; 2010, Article ID 571783:12 pages).

The antitumor activity of BTB07995 can be tested in a GW501516-dependent gastric tumor model, where tumorigenesis can be followed by MRI (Pollock C B, et al. Induction of metastatic gastric cancer by peroxisome proliferator-activated receptor-delta activation. PPAR Res. 2010; 2010, Article ID 571783:12 pages). BTB07995 can be administered by gavage at doses of 10 mg/kg and 100 mg/kg daily beginning one day after initiating the 0.005% GW501516 diet (Pollock C B, et al. Induction of metastatic gastric cancer by peroxisome proliferator-activated receptor-delta activation. PPAR Res. 2010; 2010, Article ID 571783:12 pages.). Two potential PPARγ antagonist pharmacophores have been identified, Sd-107-10 and YL-1-83, and one PPARδ antagonist, YL-1-88. Optimal potency and selectivity can be determined, as well as scale-up synthesis. Toxicology and testing of Sd-107-10 will begin as soon as scale-up synthesis of 10 g is completed.

TABLE 1
PPAR reporter assay of new analogs.

| | Binding assay | Reporter assay (% | | | | | |
|---|---|---|---|---|---|---|---|
| | (μM) | inhibition) | | | | | ID |
| | EC₅₀ | | 25 | 10 | 2.5 | 1 | |
| | | (μM) | | | | | |
| 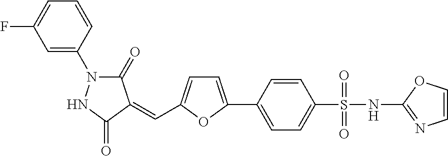 [SMILES: O=C1NN(c2cccc(F)c2)C(=O)/C1=C\c1ccc(-c2ccc(S(=O)(=O)Nc3ncco3)cc2)o1] | α nb γ 0.56 | α γ | 20 85 | 0 40 | na na | 0 0 | Sd-107-10 |
| | δ nb | δ | 10 | 0 | na | 0 | PPARγ inhibitor |
| 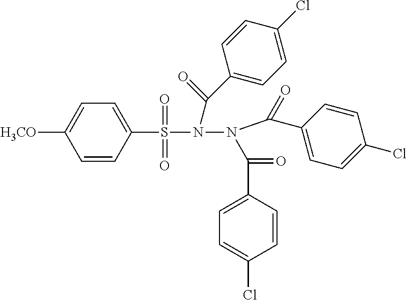 [SMILES: COc1ccc(S(=O)(=O)N(C(=O)c2ccc(Cl)cc2)N(C(=O)c2ccc(Cl)cc2)C(=O)c2ccc(Cl)cc2)cc1] | α nb γ 40.8 | α γ | 40 75 | 15 50 | 0 16 | 0 0 | YL-1-38-1 |
| | δ nb | δ | 47 | 0 | 0 | 0 | Pan inhibitor |
| 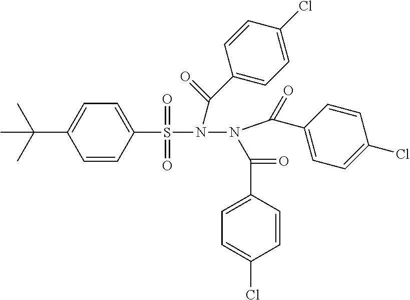 [SMILES: CC(C)(C)c1ccc(S(=O)(=O)N(C(=O)c2ccc(Cl)cc2)N(C(=O)c2ccc(Cl)cc2)C(=O)c2ccc(Cl)cc2)cc1] | α nb γ nb | α γ | 17 75 | 0 50 | na 16 | 0 0 | YL-1-68-1 |
| | δ nb | δ | 47 | 0 | 0 | 0 | PPARγ/δ inhibitor |
| 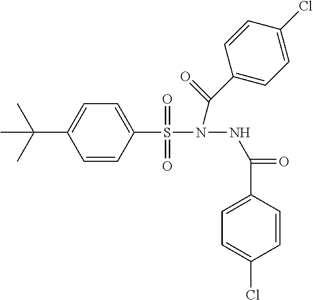 [SMILES: CC(C)(C)c1ccc(S(=O)(=O)N(NC(=O)c2ccc(Cl)cc2)C(=O)c2ccc(Cl)cc2)cc1] | α nb γ 1.4 | α γ | 57 67 | 23 23 | na na | 16 0 | YL-1-68-2 |
| | δ nb | δ | 46 | 18 | na | 0 | Pan inhibitor |
| 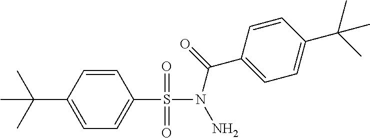 [SMILES: CC(C)(C)c1ccc(C(=O)N(N)S(=O)(=O)c2ccc(C(C)(C)C)cc2)cc1] | α nb γ nb | α γ | 20 50 | 15 25 | na na | 0 0 | YL-1-69 |
| | δ nb | δ | 20 | 10 | na | 0 | Pan inhibitor |
| 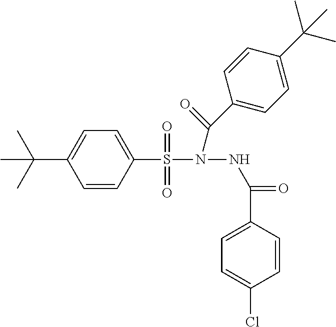 [SMILES: CC(C)(C)c1ccc(C(=O)N(NC(=O)c2ccc(Cl)cc2)S(=O)(=O)c2ccc(C(C)(C)C)cc2)cc1] | α nb γ 9.2 | α γ | 53 79 | 24 58 | na na | 0 | YL-1-80 |
| | δ nb | 18 | | | | | Pan inhibitor |
| | | δ 12 | 64 | 31 | na | | |
| 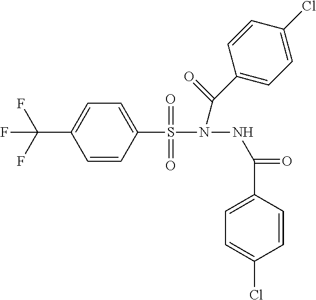 [SMILES: O=C(NN(C(=O)c1ccc(Cl)cc1)S(=O)(=O)c1ccc(C(F)(F)F)cc1)c1ccc(Cl)cc1] | α nb γ 15.2 | α γ | 8 32 | | | | YL-1-81 |
| | δ nb | δ | 46 | | | | PPARγ/δ inhibitor |
| 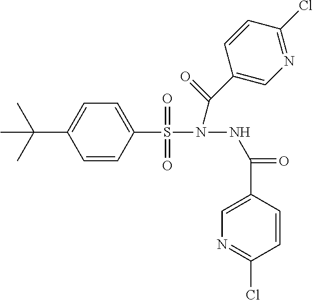 [SMILES: CC(C)(C)c1ccc(S(=O)(=O)N(NC(=O)c2ccc(Cl)nc2)C(=O)c2ccc(Cl)nc2)cc1] | α 1.3 γ 0.11 | α γ | 0 37 | 0 0 | na na | 0 0 | YL-1-83 |
| | δ nb | δ | 0 | 0 | na | 0 | PPARγ inhibitor |
| 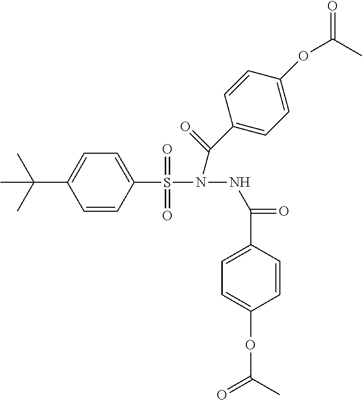 [SMILES: CC(=O)Oc1ccc(C(=O)NN(C(=O)c2ccc(OC(C)=O)cc2)S(=O)(=O)c2ccc(C(C)(C)C)cc2)cc1] | α nb γ 0.41 | α γ | 52 79 | 0 28 | na na | 0 | YL-1-87 |
| | δ nb | 19 | | | | | Pan inhibitor |
| | | δ 18 | 60 | 23 | na | | |
| 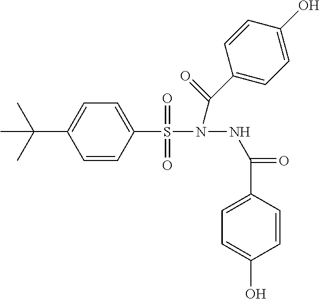 [SMILES: CC(C)(C)c1ccc(S(=O)(=O)N(NC(=O)c2ccc(O)cc2)C(=O)c2ccc(O)cc2)cc1] | α nb γ 22.3 | α γ | 0 10 | 0 0 | na na | 0 0 | YL-1-88 |
| | δ nb | δ | 40 | 14 | na | 0 | PPARδ inhibitor |

Nb, no binding;
na, not assayed

1. A compound having the structure of: 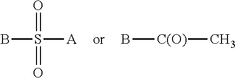
wherein:
A is: 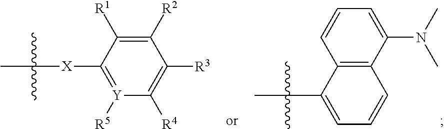
X is absent or present, if present X is —NH—;
Y is C or N, if N R⁵ is absent;
R¹, R², R³, R⁴ and R⁵ are independently hydrogen, C₁-C₃ alkyl, C₄-C₆ alkyl, C₁-C₃ alkoxy, halo, C₁-C₃ haloalkyl, cyano or nitro, wherein at least one of R¹, R², R³, R⁴ and R⁵ is not hydrogen;
B is: 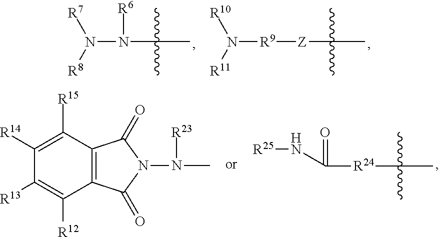
R⁶, R⁷ and R⁸ are independently hydrogen, —C(O)—CH₂—R²² or 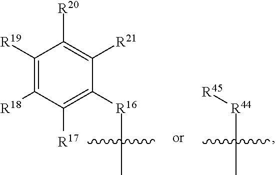
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 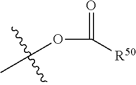
cyano or nitro, wherein at least one of R¹⁷, R¹⁸, R¹⁹, R²⁰ and R²¹ is not hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, wherein pyridine is substituted with C₁-C₆ alkyl, hydrogen, C₁-C₃ alkoxy, halo, C₁-C₃ haloalkyl, 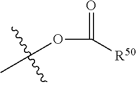
cyano or nitro,
R²² is hydroxyl, halo, or hydrogen, wherein at least one of R⁶, R⁷ and R⁸ is not hydrogen;
Z is absent or present, if present Z is —N(H)—;
R⁹ is —CH₂—, —CH₂CH₂—, —CH₂CH₂C(O)—, —CH₂C(O)—, or —C(O)—;
R¹⁰ and R¹¹ are independently hydrogen or 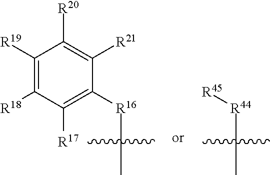
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 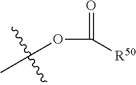
cyano or nitro, wherein at least one of R¹⁷, R¹⁸, R¹⁹, R²⁰ and R²¹ is not hydrogen, wherein R¹⁰ and R¹¹ are not both hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 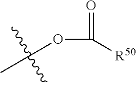
cyano or nitro;
R²³ is hydrogen or 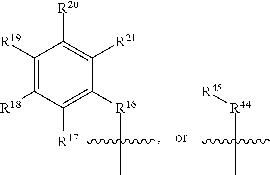
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 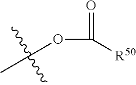
cyano or nitro, wherein at least one of R¹⁷, R¹⁸, R¹⁹, R²⁰ and R²¹ is not hydrogen
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 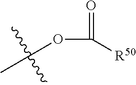
cyano or nitro,
R¹², R¹³, R¹⁴ and R¹⁵ are independently hydrogen, C₁-C₃ alkyl, C₄-C₆ alkyl, C₁-C₃ alkoxy, halo, C₁-C₃ haloalkyl, 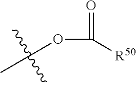
cyano or nitro, wherein at least one of R¹², R¹³, R¹⁴ and R¹⁵ is not hydrogen;
R²⁴ is —CH₂—, —CH₂CH₂—, —CH₂CH₂CH₂— or —CH₂CH₂CH₂CH₂—; and
R²⁵ is 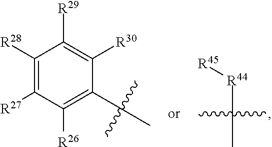
R²⁶, R²⁷, R²⁸, R²⁹ and R³⁰ are independently hydrogen, C₁-C₃ alkyl, C₄-C₆ alkyl, C₁-C₃ alkoxy, halo, C₁-C₃ haloalkyl, 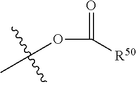
cyano or nitro, wherein at least one of R²⁶, R²⁷, R²⁸, R²⁹ and R³⁰ is not hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 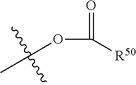
cyano or nitro; and
wherein the compound is not

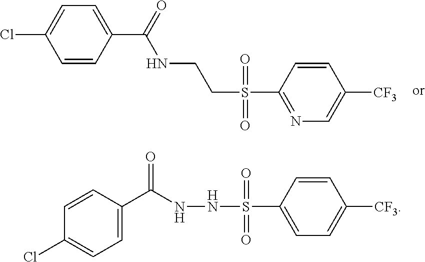 [SMILES: O=C(NCCS(=O)(=O)c1ccc(C(F)(F)F)cn1)c1ccc(Cl)cc1 ; O=C(NNS(=O)(=O)c1ccc(C(F)(F)F)cc1)c1ccc(Cl)cc1]

2-14. (canceled)

15. The compound of claim 1 having the structure:

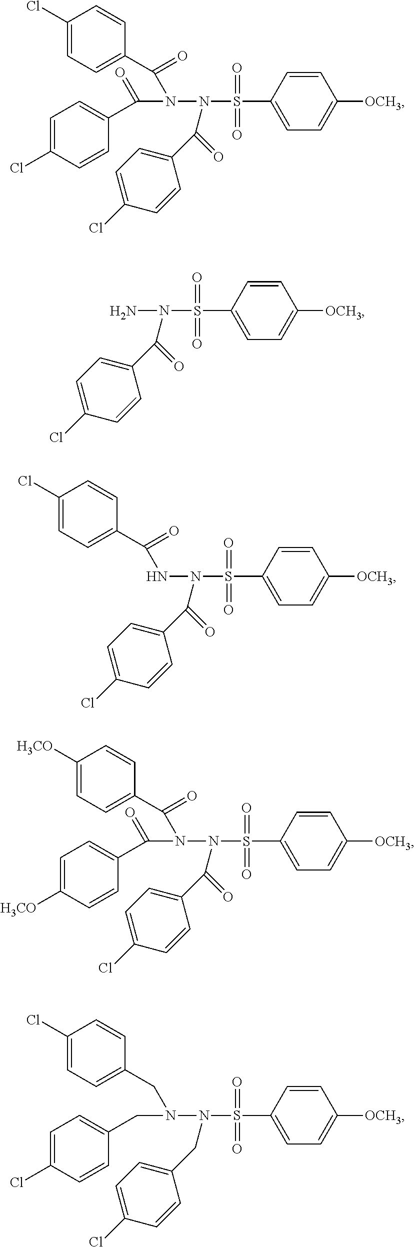

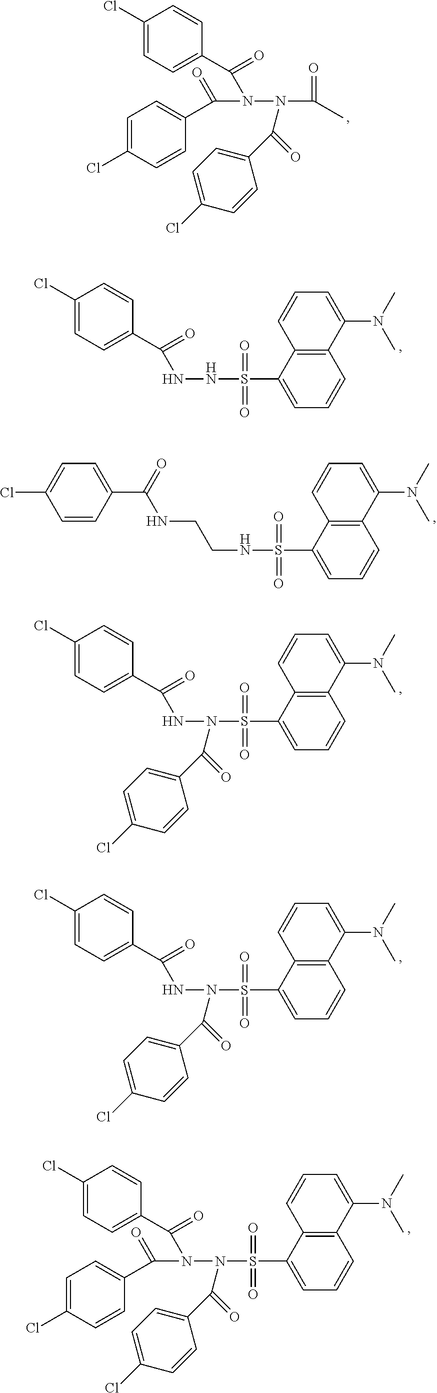 [SMILES: CC(=O)N(C(=O)c1ccc(Cl)cc1)N(C(=O)c1ccc(Cl)cc1)C(=O)c1ccc(Cl)cc1 ; CN(C)c1cccc2c(S(=O)(=O)NNC(=O)c3ccc(Cl)cc3)cccc12 ; CN(C)c1cccc2c(S(=O)(=O)NCCNC(=O)c3ccc(Cl)cc3)cccc12 ; CN(C)c1cccc2c(S(=O)(=O)N(NC(=O)c3ccc(Cl)cc3)C(=O)c3ccc(Cl)cc3)cccc12 ; CN(C)c1cccc2c(S(=O)(=O)N(NC(=O)c3ccc(Cl)cc3)C(=O)c3ccc(Cl)cc3)cccc12 ; CN(C)c1cccc2c(S(=O)(=O)N(C(=O)c3ccc(Cl)cc3)N(C(=O)c3ccc(Cl)cc3)C(=O)c3ccc(Cl)cc3)cccc12]

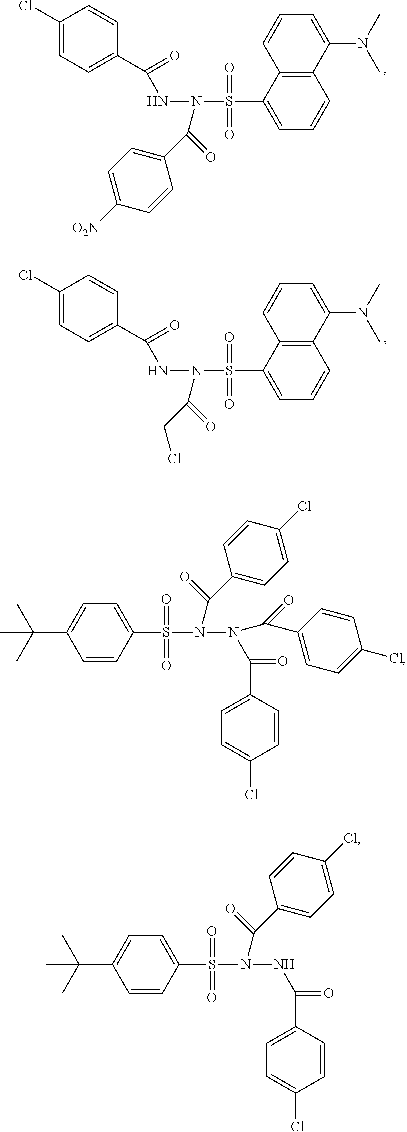

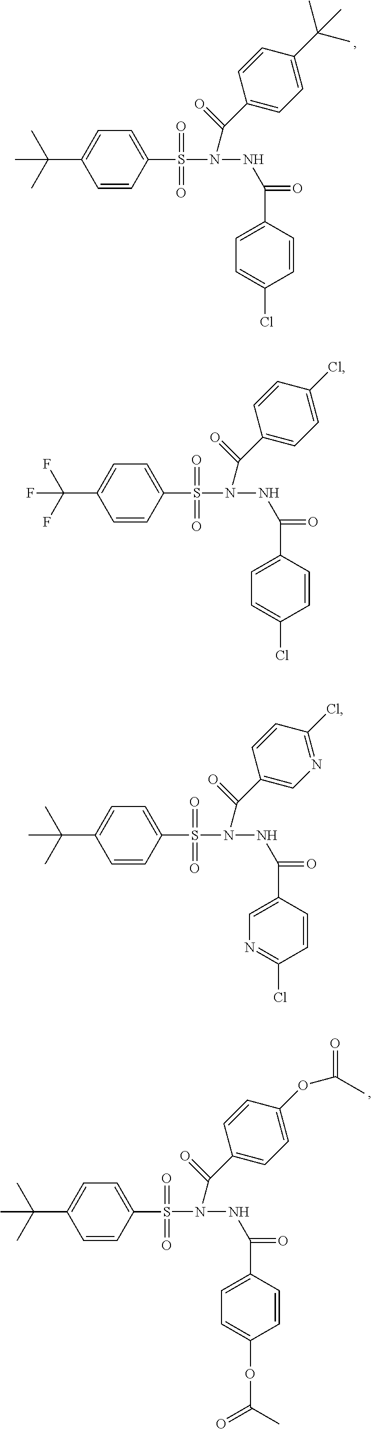

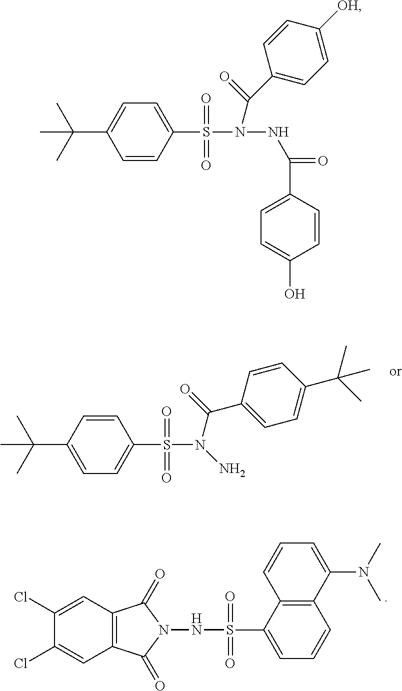 [SMILES: CC(C)(C)c1ccc(S(=O)(=O)N(NC(=O)c2ccc(O)cc2)C(=O)c2ccc(O)cc2)cc1 ; CC(C)(C)c1ccc(C(=O)N(N)S(=O)(=O)c2ccc(C(C)(C)C)cc2)cc1 ; CN(C)c1cccc2c(S(=O)(=O)NN3C(=O)c4cc(Cl)c(Cl)cc4C3=O)cccc12]

16. A compound having the structure of: 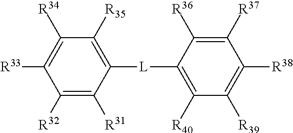
wherein:
L is —C(O)CHCH—, —C(O)(CH₂)₁₋₃—, —C(O)(CHCH)₂—, —(CHCH)₁₋₂ or —(CH₂)₁₋₄—;
R³¹, R³², R³³, R³⁴, R³⁵, R³⁶, R³⁷, R³⁸, R³⁹ or R⁴⁰ is independently hydrogen, —B(OH)₂, C₁-C₃ alkyl, C₁-C_{3 i} alkoxy, halo, C₁-C₃ haloalkyl, cyano or nitro, wherein at least four of R³¹, R³², R³³, R³⁴, R³⁵, R³⁶, R³², R³⁸, R³⁹ or R⁴⁰ are not hydrogen.

17-20. (canceled)

21. The compound of claim 16 having the structure:

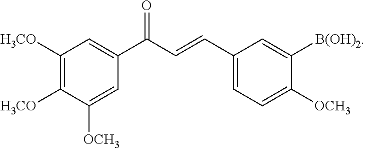 [SMILES: COc1ccc(/C=C/C(=O)c2cc(OC)c(OC)c(OC)c2)cc1B(O)O]

22. A compound having the structure of: 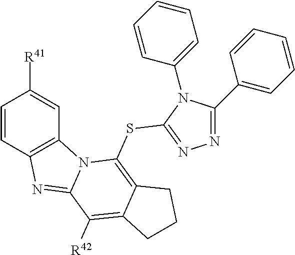
wherein:
R⁴¹ is hydrogen, hydroxyl, halo, C₁-C₃ alkyl, C₁-C₃ alkoxy, C₁-C₃ haloalkyl, nitro, cyano or —B(OH)₂;
R⁴² is hydrogen hydroxyl, halo, C₁-C₃ alkyl, C₁-C₃ alkoxy, C₁-C₃ haloalkyl, nitro, cyano, —B(OH)₂ or —C(O)—R⁴³,
R⁴³ is C₁-C₃ alkyl; and
wherein R⁴¹ and R⁴² are not both hydrogen and wherein R⁴¹ is not hydrogen if R⁴² is cyano.

23. A method of inhibiting peroxisome proliferator-activated receptors (PPAR) comprising administering a composition comprising a compound having the structure: 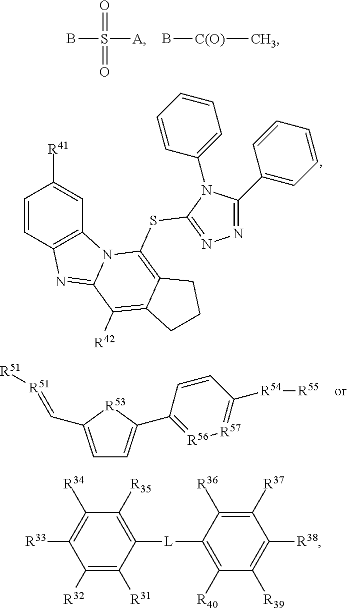
or a pharmaceutically acceptable salt, prodrug, clathrate, tautomer or solvate thereof, wherein:
A is: 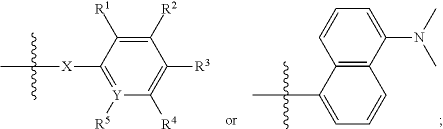
X is absent or present, if present X is —NH—;
Y is C or N, if N R⁵ is absent;
R¹, R², R³, R⁴ and R⁵ are independently hydrogen, C₁-C₃ alkyl, C₄-C₆ alkyl, C₁-C₃ alkoxy, halo, C₁-C₃ haloalkyl, cyano or nitro, wherein at least one of R¹, R², R³, R⁴ and R⁵ is not hydrogen;
B is: 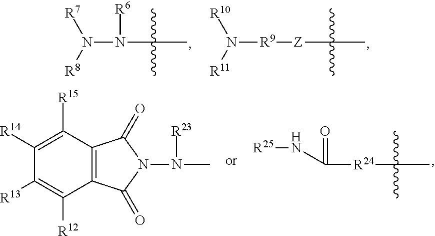
R⁶, R⁷ and R⁸ are independently hydrogen, —C(O)—CH₂—R²² or 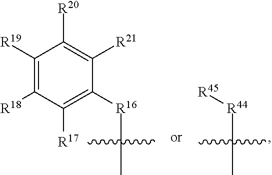
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 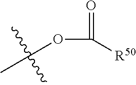
cyano or nitro, wherein at least one of R¹⁷, R¹⁸, R¹⁹, R²⁰ and R²¹ is not hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, wherein pyridine is substituted with C₁-C₆ alkyl, hydrogen, C₁-C₃ alkoxy, halo, C₁-C₃ haloalkyl, 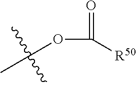
cyano or nitro,
R²² is hydroxyl, halo, or hydrogen, wherein at least one of R⁶, R⁷ and R⁸ is not hydrogen;
Z is absent or present, if present Z is —N(H)—;
R⁹ is —CH₂—, —CH₂CH₂—, —CH₂CH₂C(O)—, —CH₂C(O)—, or —C(O)—;
R¹⁰ and R¹¹ are independently hydrogen or 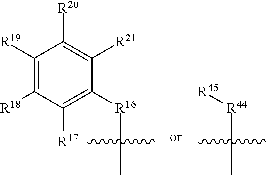
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 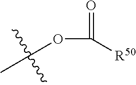
cyano or nitro, wherein at least one of R¹⁷, R¹⁸, R¹⁹, R²⁰ and R²¹ is not hydrogen, wherein R¹⁰ and R¹¹ are not both hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 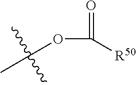
cyano or nitro;
R²³ is hydrogen or 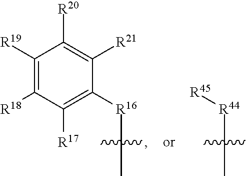
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 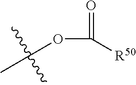
cyano or nitro, wherein at least one of R¹⁷, R¹⁸, R¹⁹, R²⁰ and R²¹ is not hydrogen
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 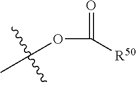
cyano or nitro,
R¹², R¹³, R¹⁴ and R¹⁵ are independently hydrogen, C₁-C₃ alkyl, C₄-C₆ alkyl, C₁-C₃ alkoxy, halo, C₁-C₃ haloalkyl, 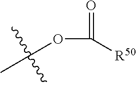
cyano or nitro, wherein at least one of R¹², R¹³, R¹⁴ and R¹⁵ is not hydrogen;
R²⁴ is —CH₂—, —CH₂CH₂—, —CH₂CH₂CH₂— or —CH₂CH₂CH₂CH₂—; and
R²⁵ is 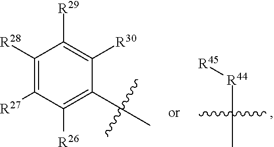
R²⁶, R²⁷, R²⁸, R²⁹ and R³⁰ are independently hydrogen, C₁-C₃ alkyl, C₄-C₆ alkyl, C₁-C₃ alkoxy, halo, C₁-C₃ haloalkyl, 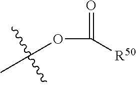
cyano or nitro, wherein at least one of R²⁶, R²⁷, R²⁸, R²⁹ and R³⁰ is not hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 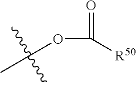
cyano or nitro;
R⁵¹ is a 5 membered heterocyclic structure having two substituents selected from ═O and ═S,
R⁵² is phenyl, ethyl, butyl, cyclohexyl, biphenyl, phenoxybenzyl propyl 1-methylcyclopropanecarboxylate or halogenated benzene,
R⁵³ is O, S or NH,
R⁵⁶ is CH and R⁵⁷ is CH, R⁵⁶ is N and R⁵⁷ is CH, or R⁵⁶ is CH and R⁵⁷ is N,
R⁵⁴ is —SO₂—, —NH—, —S(O)₂NH—, —NHCH₂—, —NHCH₂CH₂—, —NHCH₂CH₂CH₂—, —NHCOO—, —SO₂NHCOO— or —SO₂NHC(O)—,
R⁵⁵ is H, C₁-C₃ alkyl, heteroaryl, heterocyclyl, aryl or cycloalkyl;
L is —C(O)CHCH—, —C(O)(CH₂)₁₋₃—, —C(O)(CHCH)₂—, —(CHCH)₁₋₂ or —(CH₂)₁₋₄—;
R³¹, R³², R³³, R³⁴, R³⁵, R³⁶, R³⁷, R³⁸, R³⁹ or R⁴⁰ is independently hydrogen, —B(OH)₂, C₁-C₃ alkyl, C₁-C₃ alkoxy, halo, C₁-C₃ haloalkyl, cyano or nitro, wherein at least four of R³¹, R³², R³³, R³⁴, R³⁵, R³⁶, R³⁷, R³⁸, R³⁹ or R⁴⁰ are not hydrogen;
R⁴¹ is hydrogen, hydroxyl, halo, C₁-C₃ alkyl, C₁-C₃ alkoxy, C₁-C₃ haloalkyl, nitro, cyano or —B(OH)₂;
R⁴² is hydrogen hydroxyl, halo, C₁-C₃ alkyl, C₁-C₃ alkoxy, C₁-C₃ haloalkyl, nitro, cyano, —B(OH)₂ or —C(O)—R⁴³, and
R⁴³ is C₁-C₃ alkyl.

24-40. (canceled)

41. The method of claim 23, where in the compound has the structure of:

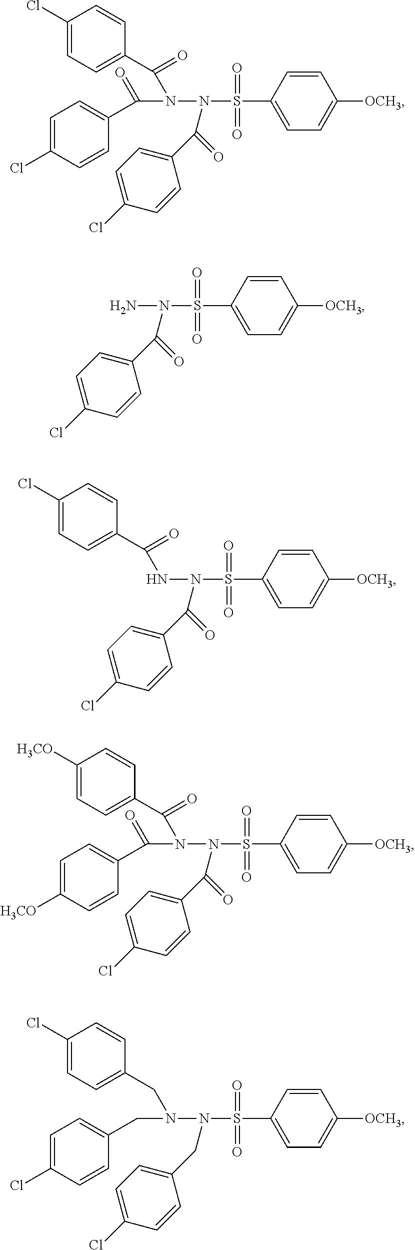

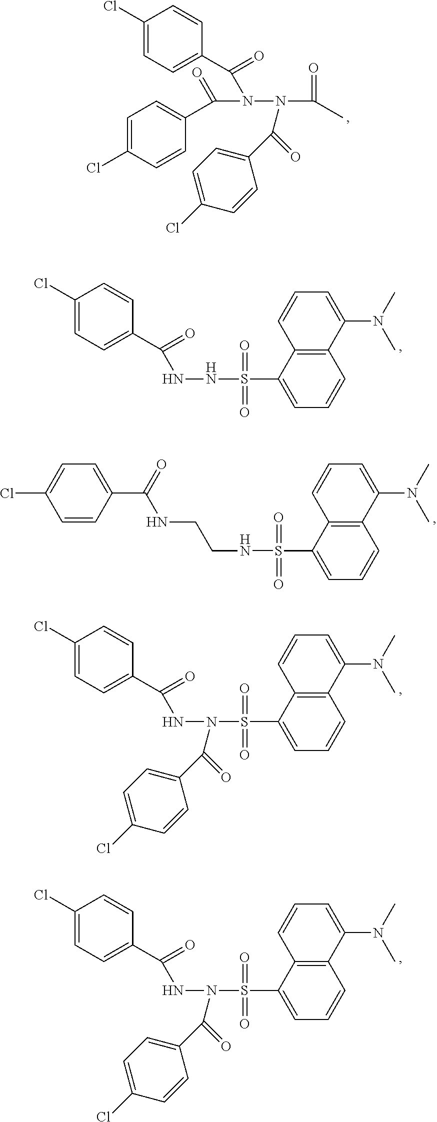 [SMILES: CC(=O)N(C(=O)c1ccc(Cl)cc1)N(C(=O)c1ccc(Cl)cc1)C(=O)c1ccc(Cl)cc1 ; CN(C)c1cccc2c(S(=O)(=O)NNC(=O)c3ccc(Cl)cc3)cccc12 ; CN(C)c1cccc2c(S(=O)(=O)NCCNC(=O)c3ccc(Cl)cc3)cccc12 ; CN(C)c1cccc2c(S(=O)(=O)N(NC(=O)c3ccc(Cl)cc3)C(=O)c3ccc(Cl)cc3)cccc12 ; CN(C)c1cccc2c(S(=O)(=O)N(NC(=O)c3ccc(Cl)cc3)C(=O)c3ccc(Cl)cc3)cccc12]

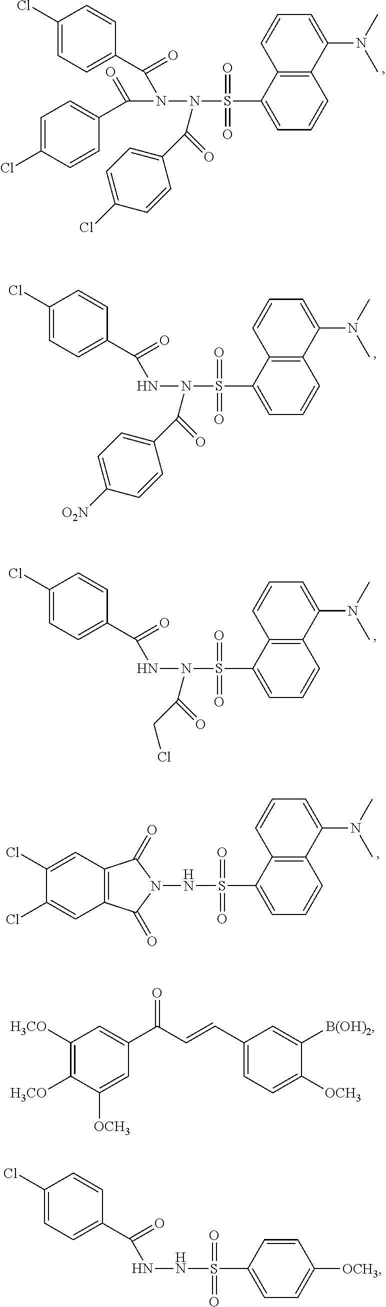 [SMILES: CN(C)c1cccc2c(S(=O)(=O)N(C(=O)c3ccc(Cl)cc3)N(C(=O)c3ccc(Cl)cc3)C(=O)c3ccc(Cl)cc3)cccc12 ; CN(C)c1cccc2c(S(=O)(=O)N(NC(=O)c3ccc(Cl)cc3)C(=O)c3ccc([N+](=O)[O-])cc3)cccc12 ; CN(C)c1cccc2c(S(=O)(=O)N(NC(=O)c3ccc(Cl)cc3)C(=O)CCl)cccc12 ; CN(C)c1cccc2c(S(=O)(=O)NN3C(=O)c4cc(Cl)c(Cl)cc4C3=O)cccc12 ; COc1ccc(/C=C/C(=O)c2cc(OC)c(OC)c(OC)c2)cc1B(O)O ; COc1ccc(S(=O)(=O)NNC(=O)c2ccc(Cl)cc2)cc1]

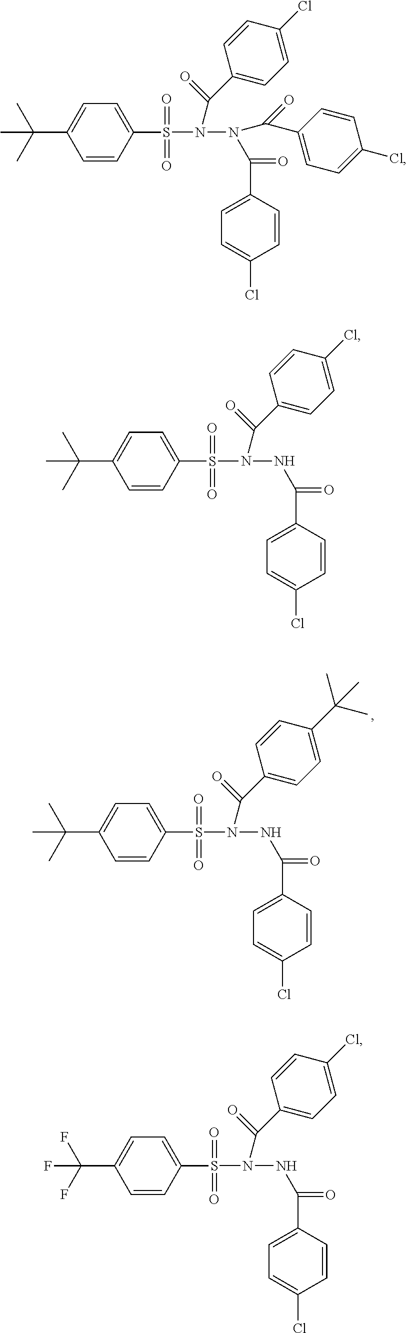 [SMILES: CC(C)(C)c1ccc(S(=O)(=O)N(C(=O)c2ccc(Cl)cc2)N(C(=O)c2ccc(Cl)cc2)C(=O)c2ccc(Cl)cc2)cc1 ; CC(C)(C)c1ccc(S(=O)(=O)N(NC(=O)c2ccc(Cl)cc2)C(=O)c2ccc(Cl)cc2)cc1 ; CC(C)(C)c1ccc(C(=O)N(NC(=O)c2ccc(Cl)cc2)S(=O)(=O)c2ccc(C(C)(C)C)cc2)cc1 ; O=C(NN(C(=O)c1ccc(Cl)cc1)S(=O)(=O)c1ccc(C(F)(F)F)cc1)c1ccc(Cl)cc1]

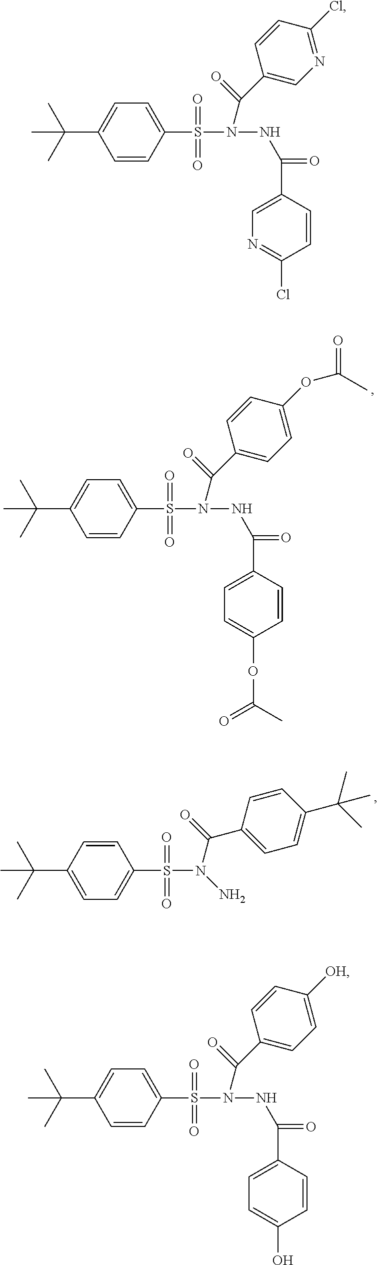

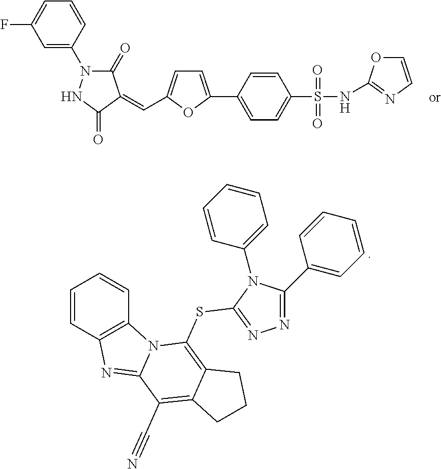 [SMILES: O=C1NN(c2cccc(F)c2)C(=O)/C1=C\c1ccc(-c2ccc(S(=O)(=O)Nc3ncco3)cc2)o1 ; N#Cc1c2c(c(Sc3nnc(-c4ccccc4)n3-c3ccccc3)n3c1nc1ccccc13)CCC2]

42. The method of claim 23, wherein the PPAR is PPARγ.

43. A method of treating cancer comprising administering a composition comprising a compound having the structure: 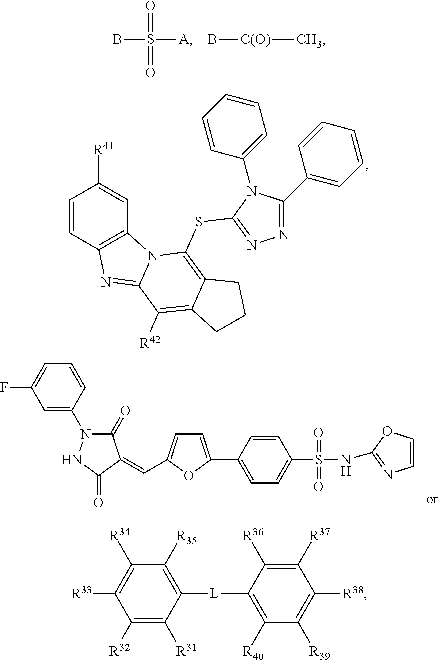
or a pharmaceutically acceptable salt, prodrug, clathrate, tautomer or solvate thereof, wherein:
A is: 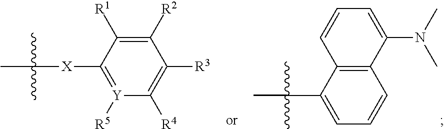
X is absent or present, if present X is —NH—;
Y is C or N, if N R⁵ is absent;
R¹, R², R³, R⁴ and R⁵ are independently hydrogen, C₁-C₃ alkyl, C₄-C₆ alkyl, C₁-C₃ alkoxy, halo, C₁-C₃ haloalkyl, cyano or nitro, wherein at least one of R¹, R², R³, R⁴ and R⁵ is not hydrogen;
B is: 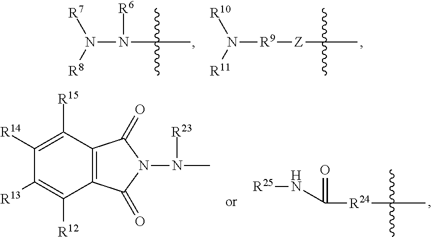
R⁶, R⁷ and R⁸ are independently hydrogen, —C(O)—CH₂—R²² or 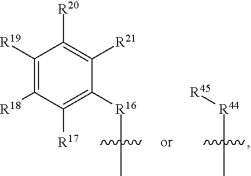
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 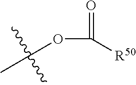
cyano or nitro, wherein at least one of R¹⁷, R¹⁸, R¹⁹, R²⁰ and R²¹ is not hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, wherein pyridine is substituted with C₁-C₆ alkyl, hydrogen, C₁-C₃ alkoxy, halo, C₁-C₃ haloalkyl, 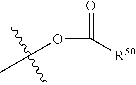
cyano or nitro,
R²² is hydroxyl, halo, or hydrogen, wherein at least one of R⁶, R⁷ and R⁸ is not hydrogen;
Z is absent or present, if present Z is —N(H)—;
R⁹ is —CH₂—, —CH₂CH₂—, —CH₂CH₂C(O)—, —CH₂C(O)—, or —C(O)—;
R¹⁰ and R¹¹ are independently hydrogen or 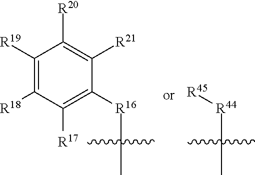
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 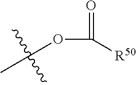
cyano or nitro, wherein at least one of R¹⁷, R¹⁸, R¹⁹, R²⁰ and R²¹ is not hydrogen, wherein R¹⁰ and R¹¹ are not both hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 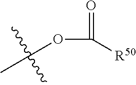
cyano or nitro;
R²³ is hydrogen or 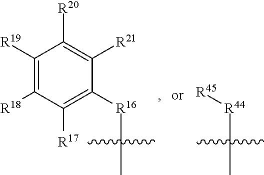
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 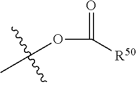
cyano or nitro, wherein at least one of R¹⁷, R¹⁸, R¹⁹, R²⁰ and R²¹ is not hydrogen
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 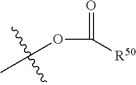
cyano or nitro,
R¹², R¹³, R¹⁴ and R¹⁵ are independently hydrogen, C₁-C₃ alkyl, C₄-C₆ alkyl, C₁-C₃ alkoxy, halo, C₁-C₃ haloalkyl, 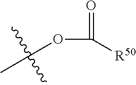
cyano or nitro, wherein at least one of R¹², R¹³, R¹⁴ and R¹⁵ is not hydrogen;
R²⁴ is —CH₂—, —CH₂CH₂—, —CH₂CH₂CH₂— or —CH₂CH₂CH₂CH₂—; and
R²⁵ is 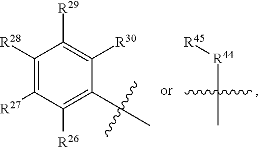
R²⁶, R²⁷, R²⁸, R²⁹ and R³⁰ are independently hydrogen, C₁-C₃ alkyl, C₄-C₆ alkyl, C₁-C₃ alkoxy, halo, C₁-C₃ haloalkyl, 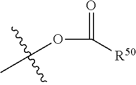
cyano or nitro, wherein at least one of R²⁶, R²⁷, R²⁸, R²⁹ and R³⁰ is not hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 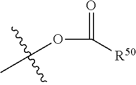
cyano or nitro;
R⁵¹ is a 5 membered heterocyclic structure having two substituents selected from ═O and ═S,
R⁵² is phenyl, ethyl, butyl, cyclohexyl, biphenyl, phenoxybenzyl propyl 1-methylcyclopropanecarboxylate or halogenated benzene,
R⁵³ is O, S or NH,
R⁵⁶ is CH and R⁵⁷ is CH, R⁵⁶ is N and R⁵⁷ is CH, or R⁵⁶ is CH and R⁵⁷ is N,
R⁵⁴ is —SO₂—, —NH—, —S(O)₂NH—, —NHCH₂—, —NHCH₂CH₂—, —NHCH₂CH₂CH₂—, —NHCOO—, —SO₂NHCOO— or —SO₂NHC(O)—,
R⁵⁵ is H, C₁-C₃ alkyl, heteroaryl, heterocyclyl, aryl or cycloalkyl;
L is —C(O)CHCH—, —C(O)(CH₂)₁₋₃—, —C(O)(CHCH)₂—, —(CHCH)₁₋₂ or —(CH₂)₁₋₄—;
R³¹, R³², R³³, R³⁴, R³⁵, R³⁶, R³⁷, R³⁸, R³⁹ or R⁴⁰ is independently hydrogen, —B(OH)₂, C₁-C₃ alkyl, C₁-C₃ alkoxy, halo, C₁-C₃ haloalkyl, cyano or nitro, wherein at least four of R³¹, R³², R³³, R³⁴, R³⁵, R³⁶, R³⁷, R³⁸, R³⁹ or R⁴⁰ are not hydrogen;
R⁴¹ is hydrogen, hydroxyl, halo, C₁-C₃ alkyl, C₁-C₃ alkoxy, C₁-C₃ haloalkyl, nitro, cyano or —B(OH)₂;
R⁴² is hydrogen hydroxyl, halo, C₁₃-C₃ alkyl, C₁-C₃ alkoxy, C₁-C₃ haloalkyl, nitro, cyano, —B(OH)₂ or —C(O)—R⁴³, and
R⁴³ is C₁-C₃ alkyl.

44. The method of claim 43, wherein the composition induces estrogen receptor alpha (ERα) expression in cancer cells.

45. The method of claim 44, wherein the cancer cells are ERα negative.

46. The method of claim 44, wherein the ERα expression results in ERα dependent cancer cells.

47. The method of claim 46, wherein the ERα dependent cancer cells are responsive to anti-estrogen therapy.

48. The method of claim 47 further comprising administering an anti-estrogen therapy.

49. A method of treating metabolic disorders comprising administering a composition comprising a compound having the structure: 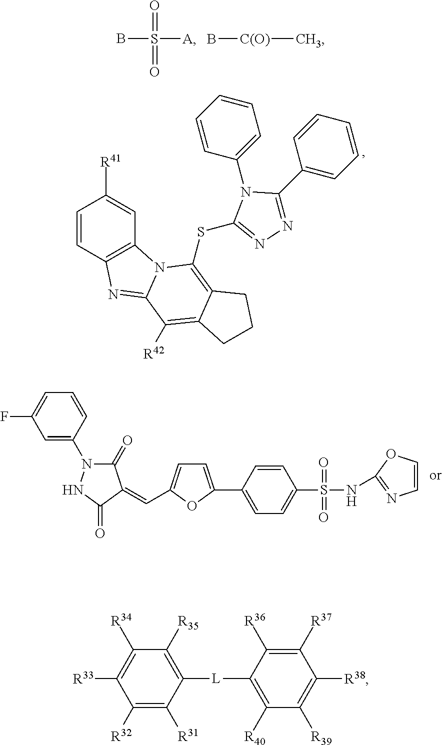
or a pharmaceutically acceptable salt, prodrug, clathrate, tautomer or solvate thereof, wherein:
A is: 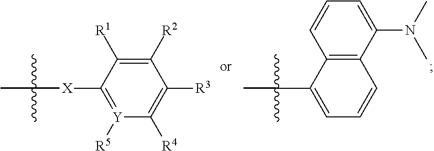
X is absent or present, if present X is —NH—;
Y is C or N, if N R⁵ is absent;
R¹, R², R³, R⁴ and R⁵ are independently hydrogen, C₁-C₃ alkyl, C₄-C₆ alkyl, C₁-C₃ alkoxy, halo, C₁-C₃ haloalkyl, cyano or nitro, wherein at least one of R¹, R², R³, R⁴ and R⁵ is not hydrogen;
B is: 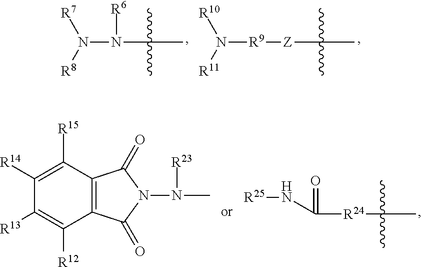
R⁶, R⁷ and R⁸ are independently hydrogen, —C(O)—CH₂—R²² or 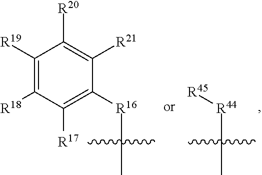
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 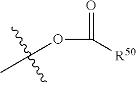
cyano or nitro, wherein at least one of R¹⁷, R¹⁸, R¹⁹, R²⁰ and R²¹ is not hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, wherein pyridine is substituted with C₁-C₆ alkyl, hydrogen, C₁-C₃ alkoxy, halo, C₁-C₃ haloalkyl, 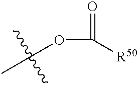
cyano or nitro,
R²² is hydroxyl, halo, or hydrogen, wherein at least one of R⁶, R⁷ and R⁸ is not hydrogen;
Z is absent or present, if present Z is —N(H)—;
R⁹ is —CH₂—, —CH₂CH₂—, —CH₂CH₂C(O)—, —CH₂C(O)—, or —C(O)—;
R¹⁰ and R¹¹ are independently hydrogen or 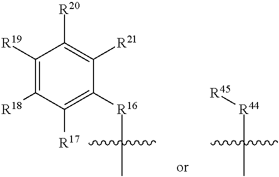
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 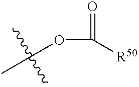
cyano or nitro, wherein at least one of R¹⁷, R¹⁸, R¹⁹, R²⁰ and R²¹ is not hydrogen, wherein R¹⁰ and R¹¹ are not both hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 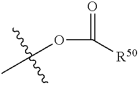
cyano or nitro;
R²³ is hydrogen or 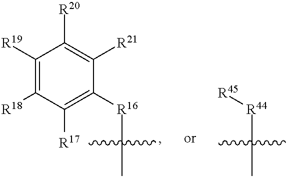
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 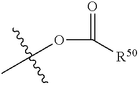
cyano or nitro, wherein at least one of R¹⁷, R¹⁸, R¹⁹, R²⁰ and R²¹ is not hydrogen
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 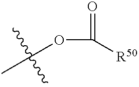
cyano or nitro,
R¹², R¹³, R¹⁴ and R¹⁵ are independently hydrogen, C₁-C₃ alkyl, C₄-C₆ alkyl, C₁-C₃ alkoxy, halo, C₁-C₃ haloalkyl, 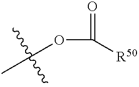
cyano or nitro, wherein at least one of R¹², R¹³, R¹⁴ and R¹⁵ is not hydrogen;
R²⁴ is —CH₂—, —CH₂CH₂—, —CH₂CH₂CH₂— or —CH₂CH₂CH₂CH₂—; and
R²⁵ is 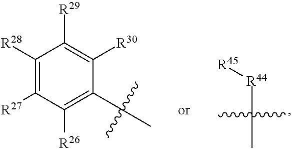
R²⁶, R²⁷, R²⁸, R²⁹ and R³⁰ are independently hydrogen, C₁-C₃ alkyl, C₄-C₆ alkyl, C₁-C₃ alkoxy, halo, C₁-C₃ haloalkyl, 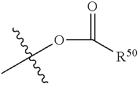
cyano or nitro, wherein at least one of R²⁶, R²⁷, R²⁸, R²⁹ and R³⁰ is not hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 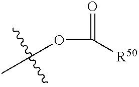
cyano or nitro;
R⁵¹ is a 5 membered heterocyclic structure having two substituents selected from ═O and ═S,
R⁵² is phenyl, ethyl, butyl, cyclohexyl, biphenyl, phenoxybenzyl propyl 1-methylcyclopropanecarboxylate or halogenated benzene,
R⁵³ is O, S or NH,
R⁵⁶ is CH and R⁵⁷ is CH, R⁵⁶ is N and R⁵⁷ is CH, or R⁵⁶ is CH and R⁵⁷ is N,
R⁵⁴ is —SO₂—, —NH—, —S(O)₂NH—, —NHCH₂—, —NHCH₂CH₂—, —NHCH₂CH₂CH₂—, —NHCOO—, —SO₂NHCOO— or —SO₂NHC(O)—,
R⁵⁵ is H, C₁-C₃ alkyl, heteroaryl, heterocyclyl, aryl or cycloalkyl;
L is —C(O)CHCH—, —C(O)(CH₂)₁₋₃—, —C(O)(CHCH)₂—, —(CHCH)₁₋₂ or —(CH₂)₁₋₄—;
R³¹, R³², R³³, R³⁴, R³⁵, R³⁶, R³⁷, R³⁸, R³⁹ or R⁴⁰ is independently hydrogen, —B(OH)₂, C₁-C₃ alkyl, C₁-C₃ alkoxy, halo, C₁-C₃ haloalkyl, cyano or nitro, wherein at least four of R³¹, R³², R³³, R³⁴, R³⁵, R³⁶, R³⁷, R³⁸, R³⁹ or R⁴⁰ are not hydrogen;
R⁴¹ is hydrogen, hydroxyl, halo, C₁-C₃ alkyl, C₁-C₃ alkoxy, C₁-C₃ haloalkyl, nitro, cyano or —B(OH)₂;
R⁴² is hydrogen hydroxyl, halo, C₁-C₃ alkyl, C₁-C₃ alkoxy, C₁-C₃ haloalkyl, nitro, cyano, —B(OH)₂ or —C(O)—R⁴³, and
R⁴³ is C₁-C₃ alkyl.

50. The method of claim 49, wherein the metabolic disorder is dislipidemia or diabetes.

51. A method of preventing or treating a PPAR-mediated disease or condition comprising administering a therapeutically effective amount of a composition comprising a compound having the structure: 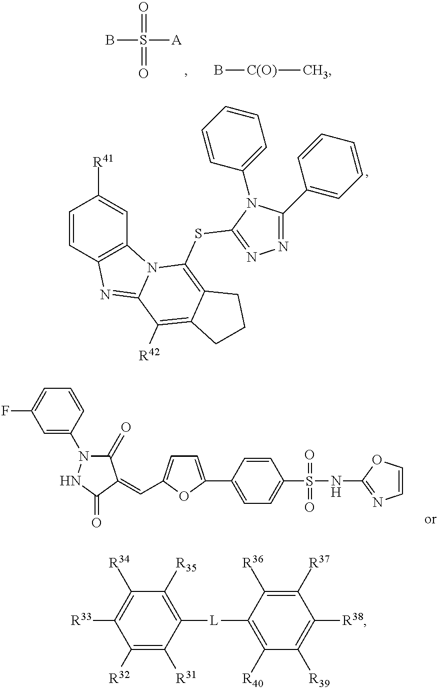
or a pharmaceutically acceptable salt, prodrug, clathrate, tautomer or solvate thereof, wherein:
A is: 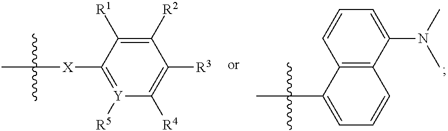
X is absent or present, if present X is —NH—;
Y is C or N, if N R⁵ is absent;
R¹, R², R³, R⁴ and R⁵ are independently hydrogen, C₁-C₃ alkyl, C₄-C₆ alkyl, C₁-C₃ alkoxy, halo, C₁-C₃ haloalkyl, cyano or nitro, wherein at least one of R¹, R², R³, R⁴ and R⁵ is not hydrogen;
B is: 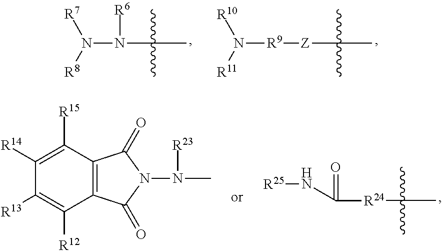
R⁶, R⁷ and R⁸ are independently hydrogen, —C(O)—CH₂—R²² or 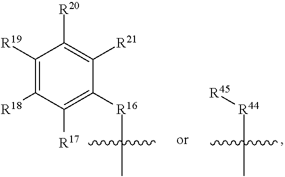
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 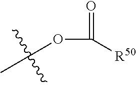
cyano or nitro, wherein at least one of R¹⁷, R¹⁸, R¹⁹, R²⁰ and R²¹ is not hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, wherein pyridine is substituted with C₁-C₆ alkyl, hydrogen, C₁-C₃ alkoxy, halo, C₁-C₃ haloalkyl, 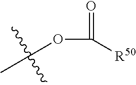
cyano or nitro,
R²² is hydroxyl, halo, or hydrogen, wherein at least one of R⁶, R⁷ and R⁸ is not hydrogen;
Z is absent or present, if present Z is —N(H)—;
R⁹ is —CH₂—, —CH₂CH₂—, —CH₂CH₂C(O)—, —CH₂C(O)—, or —C(O)—;
R¹⁰ and R¹¹ are independently hydrogen or 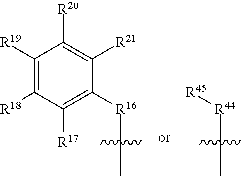
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 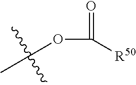
cyano or nitro, wherein at least one of R¹⁷, R¹⁸, R¹⁹, R²⁰ and R²¹ is not hydrogen, wherein R¹⁰ and R¹¹ are not both hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 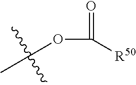
cyano or nitro;
R²³ is hydrogen or 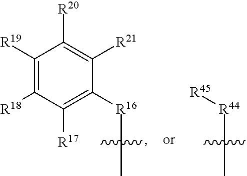
R¹⁶ is —CH₂—, —CH₂CH₂—, —CH₂CH₂C(O)—, —CH₂C(O)—, or —C(O)—,
R¹⁷, R¹⁸, R¹⁹, R²⁰ and R²¹ are independently hydrogen, C₁-C₃ alkyl, C₄-C₆ alkyl, C₁-C₃ alkoxy, halo, C₁-C₃ haloalkyl, 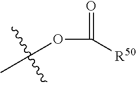
cyano or nitro, wherein at least one of R¹⁷, R¹⁸, R¹⁹, R²⁰ and R²¹ is not hydrogen
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 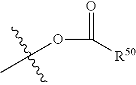
cyano or nitro,
R¹², R¹³, R¹⁴ and R¹⁵ are independently hydrogen, C₁-C₃ alkyl, C₄-C₆ alkyl, C₁-C₃ alkoxy, halo, C₁-C₃ haloalkyl, 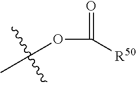
cyano or nitro, wherein at least one of R¹², R¹³, R¹⁴ and R¹⁵ is not hydrogen;
R²⁴ is —CH₂—, —CH₂CH₂—, —CH₂CH₂CH₂— or —CH₂CH₂CH₂CH₂—; and
R²⁵ is 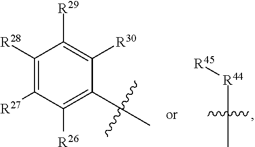
R²⁶, R²⁷, R²⁸, R²⁹ and R³⁰ are independently hydrogen, C₁-C₃ alkyl, C₄-C₆ alkyl, C₁-C₃ alkoxy, halo, C₁-C₃ haloalkyl, 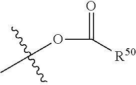
cyano or nitro, wherein at least one of R²⁶, R²⁷, R²⁸, R²⁹ and R³⁰ is not hydrogen,
R⁵⁰ is H or C₁-C₆ alkyl,
R⁴⁴ is —CH₂—, —CH₂CH₂—, —CH₂CH₂C(O)—, —CH₂C(O)—, or —C(O)—,
R⁴⁵ is substituted pyridine, substituted with C₁-C₆ alkyl, hydrogen, C₁-C₃ alkoxy, halo, C₁-C₃ haloalkyl, 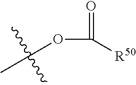
cyano or nitro;
R⁵¹ is a 5 membered heterocyclic structure having two substituents selected from ═O and ═S,
R⁵² is phenyl, ethyl, butyl, cyclohexyl, biphenyl, phenoxybenzyl propyl 1-methylcyclopropanecarboxylate or halogenated benzene,
R⁵³ is O, S or NH,
R⁵⁶ is CH and R⁵⁷ is CH, R⁵⁶ is N and R⁵⁷ is CH, or R⁵⁶ is CH and R⁵⁷ is N,
R⁵⁴ is —SO₂—, —NH—, —S(O)₂NH—, —NHCH₂—, —NHCH₂CH₂—, —NHCH₂CH₂CH₂—, —NHCOO—, —SO₂NHCOO— or —SO₂NHC(O)—,
R⁵⁵ is H, C₁-C₃ alkyl, heteroaryl, heterocyclyl, aryl or cycloalkyl;
L is —C(O)CHCH—, —C(O)(CH₂)₁₋₃—, —C(O)(CHCH)₂—, —(CHCH)₁₋₂ or —(CH₂)₁₋₄—;
R³¹, R³², R³³, R³⁴, R³⁵, R³⁶, R³⁷, R³⁸, R³⁹ or R⁴⁰ is independently hydrogen, —B(OH)₂, C₁-C₃ alkyl, C₁-C_{3 i} alkoxy, halo, C₁-C₃ haloalkyl, cyano or nitro, wherein at least four of R³¹, R³², R³³, R³⁴, R³⁵, R³⁶, R³⁷, R³⁸, R³⁹ or R⁴⁰ are not hydrogen;
R⁴¹ is hydrogen, hydroxyl, halo, C₁-C₃ alkyl, C₁-C₃ alkoxy, C₁-C₃ haloalkyl, nitro, cyano or —B(OH)₂;
R⁴² is hydrogen hydroxyl, halo, C₁-C₃ alkyl, C₁-C₃ alkoxy, C₁-C₃ haloalkyl, nitro, cyano, —B(OH)₂ or —C(O)—R⁴³, and
R⁴³ is C₁-C₃ alkyl.

52. The method of claim 51, wherein the compound is

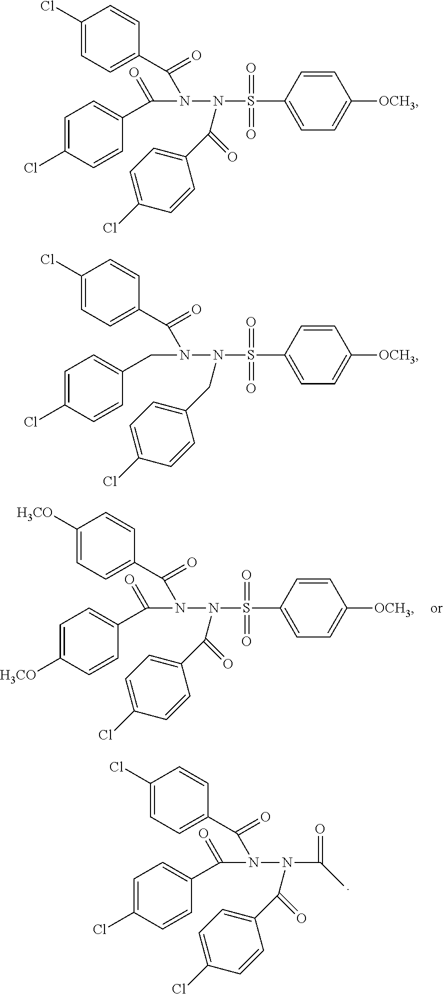

53. The method of claim 51, wherein the PPAR-mediated disease or condition is a PPARγ-mediated disease or condition, wherein the disease or condition is selected from the group consisting of diabetes, obesity, metabolic syndrome, impaired glucose tolerance, syndrome X, and cardiovascular disease, or both.

54. (canceled)

55. The method of claim 51, wherein the disease or condition is selected from the group consisting of diabetes and cardiovascular disease.

56. A compound having the structure of: 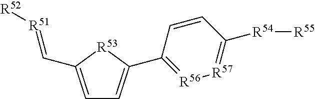
wherein:
R⁵¹ is 5 membered heterocyclic structure having two substituents selected from ═O and ═S,
R⁵² is substituted or unsubstituted aryl, substituted or unsubstituted heteroaryl, substituted or unsubstituted cycloalkyl, substituted or unsubstituted heterocyclyl, 1-methylcyclopropanecarboxylate C₁-C₆ alkyl, 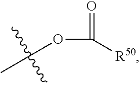
C₁-C₃ alkoxy, halo, C₁-C₃ haloalkyl, cyano or nitro,
R⁵⁰ is C₁-C₆ alkyl,
R⁵³ is O, S or NH,
R⁵⁶ is CH and R⁵⁷ is CH, R⁵⁶ is N and R⁵⁷ is CH, or R⁵⁶ is CH and R⁵⁷ is N,
R⁵⁴ is —SO₂—, —NH—, —S(O)₂NH—, —NHCH₂—, —NHCH₂CH₂—, —NHCH₂CH₂CH₂—, —NHCOO—, —SO₂NHCOO— or —SO₂NHC(O)—,
R⁵⁵ is H, C₁-C₃ alkyl, heteroaryl, heterocyclyl, aryl or cycloalkyl.

57. The compound of claim 56, wherein R⁵¹ is pyrazolidine-3,5,dione, 2-thioxothiazolidin-4-1, 2-thioxooxazolidin-4-1, thiazolidine-2,4-dione or 5-thioxopyrazolidin-3-1, R⁵² is phenyl, ethyl, butyl, cyclohexyl, biphenyl, phenoxybenzyl propyl 1-methylcyclopropanecarboxylate or halogenated benzene, R⁵³ is O, S, or NH, R⁵⁶ is CH and R⁵⁷ is CH, R⁵⁴ is —SO₂—, —NH— or —S(O)₂NH—, and R⁵⁵ is H, C₁-C₃ alkyl, phenyl, pyrrole imidazole, oxazole, thiazole or triazole.

58. The compound of claim 57, wherein R⁵¹ is pyrazolidine-3,5,dione, R⁵² is halogenated benzene, R⁵³ is O, R⁵⁶ is CH and R⁵⁷ is CH, R⁵⁴ is —S(O)₂NH—, and R⁵⁵ is phenyl, pyrrole imidazole, oxazole, thiazole or triazole.
